(19) United States
(12) Patent Application Publication
(10) Pub. No.: US 2025/0177558 A1
(43) Pub. Date: Jun. 5, 2025

(54) Functionalized Nanoclusters and Their Use in Treating Bacterial Infections

(71) Applicant: The Board of Trustees of the Leland Stanford Junior University, Stanford, CA (US)

(72) Inventors: Peter Luke Santa Maria, Emerald Hills, CA (US); Laurent Bekale, Redwood City, CA (US)

(21) Appl. No.: 18/840,401

(22) PCT Filed: Feb. 27, 2023
(86) PCT No.: PCT/US2023/013965

§ 371 Date: Aug. 21, 2024

Related U.S. Application Data
(60) Provisional application No. 63/314,642, filed on Feb. 28, 2022.

(51) Int. Cl. A61K 47/69; A61K 33/242; A61K 47/64; A61P 31/04

(52) U.S. Cl. CPC A61K 47/6923; A61K 33/242; A61K 47/64; A61P 31/04

(57) ABSTRACT

Compositions, methods, and kits are provided for treating bacterial infections with nanoclusters comprising a metallic core conjugated to a nucleotide. Recalcitrant infections are often difficult to treat because of the presence of persister cells, a subpopulation of bacterial cells that is highly tolerant of traditional antibiotics. Persister cells are dormant, which makes them less susceptible to many antibiotics, which are designed to kill growing cells. Administration of nanoclusters comprising a nucleotide was found to be highly efficacious in eradicating persister cells and for treating infections for a broad range of bacterial species, including Gram-positive and Gram-negative bacteria. Such treatment was effective not only in eradicating planktonic bacteria but also bacteria in biofilms.

### STATEMENT REGARDING FEDERALLY SPONSORED RESEARCH OR DEVELOPMENT

This invention was made with Government support under contract AI154097 awarded by the National Institutes of Health. The Government has certain rights in the invention.

### BACKGROUND

Antibiotics are the mainstay of modern clinical medicine. However, bacteria develop resistance to both natural and synthetic antibiotics within years of their first clinical use (Walsh (2003) Nature Reviews Microbiology 1:65-70). Current mechanisms of antibiotic resistance include: decreased uptake by changes in outer membrane permeability; antibiotic excretion by activation of efflux pump-proteins; enzymatic modification of the antibiotic; modification of antibiotic targets; and bacterial physiology such as biofilm (van Hoek et al. (2011) Front Microbiol 2:203).

In the United States and Europe alone, over 50,000 people die every year because of resistant infections (The Review on Antimicrobial Resistance. Antimicrobial Resistance: Tackling a crisis wealth nations for the health and of (2014), amr-review.org/Publications.html)). Lengths of stays in a hospital are prolonged by antibiotic-resistant infections, and these same infections are often acquired in hospitals. The economic impact of antibiotic resistant infections is estimated to be between US $5 billion and US $24 billion per year in the United States alone (Hall (2004) Nature Reviews Microbiology 2:430-435). However, the drug pipelines of pharmaceutical companies have not kept pace with the evolution of antibiotic resistance. In 2004, only 1.5% of all the drugs in development by the world's 15 largest pharmaceutical companies were antibiotics (Smith and Coast, “The economic burden of antimicrobial resistance: why it is more serious than current studies suggest.” (2012), researchgate.net/publication/291413454). The new reality that we must face is that the pharmaceutical companies are not presently aligned for the discovery of new antibiotics. A strategy to protect our existing antibiotics is through the use of antibiotic adjuvants, compounds that enhance the activity of current drugs and minimize, and even directly block resistance (Lu et al. (2009) Proc. Natl. Acad. Sci. U.S.A. 106 (12): 4629-4634, Gonzalez-Bello (2017) Bioorg. Med. Chem. Lett. 27 (18): 4221-4228). Another strategy is the used of ant-virulence agents. These agents can circumvent antibiotic resistance by disarming pathogens of virulence factors that facilitate human disease while leaving bacterial growth pathways (Dickey et al. (2017) Nat. Rev. Drug Discov. 16 (7): 457-471).

Bacterial cells, attached to a surface, can aggregate to each other to form biofilms. Bacteria growing biofilms may exhibit increased tolerance to antimicrobial agents, it is very difficult or eliminate substantially reduce. Biofilm bacteria have two dormant phenotypes: the viable but non-culturable (VBNC) state and the persister state. Dormant phenotypes (VBNC and persisters) allow bacteria to survive in conditions that are deadly to the rest of their genetically identical lineage. Once in biofilms, they can escape the immune system. Thus, one of the main roles of biofilm is to provide a protective habitat for persisters and VBNC by shielding them from the immune system (Lewis (2010) Microbe (Washington, D.C.) 5 (10): 429-437). Another property of biofilms is their capacity to be more resistant to antimicrobial agents than planktonic cells (Spoering et al. (2001) J. Bacteriol. 183 (23): 6746-6751). Thus, there is an ongoing and unmet need for an improved approach for treating antibiotic resistant infections.

### SUMMARY

Compositions, methods, and kits are provided for treating bacterial infections with nanoclusters comprising a metallic core conjugated to a nucleotide. Recalcitrant infections are often difficult to treat because of the presence of persister cells, a subpopulation of bacterial cells that is highly tolerant of traditional antibiotics. Persister cells are dormant, which makes them less susceptible to many antibiotics, which are designed to kill growing cells. Administration of nanoclusters comprising a nucleotide was found to be highly efficacious in eradicating persister cells and for treating infections for a broad range of bacterial species, including Gram-positive and Gram-negative bacteria. Such treatment was effective not only in eradicating planktonic bacteria but also bacteria in biofilms.

In one aspect, a nanocluster is provided comprising a metallic core conjugated to a nucleotide. In some embodiments, the metallic core comprises a noble metal. In some embodiments, the nanocluster comprises a gold metallic core. In some embodiments, the nanocluster is biocompatible with human cells.

In certain embodiments, the nucleotide is adenosine triphosphate (ATP) or a phosphorothioate analog, a deoxyribonucleotide analog, a 7-deaza purine nucleotide analog, or a phosphomethylphosphonic acid adenylate ester thereof. Exemplary, phosphorothioate analogs include, without limitation, ATPaS, ATPBS, or ATPyS. Exemplary deoxyribonucleotide analogs include, without limitation, deoxyadenosine triphosphate (dATP). Exemplary 7-deaza purine nucleotide analogs include, without limitation, 7-deazaadenosine-5′-triphosphate (7-deaza-ATP). Exemplary phosphomethylphosphonic acid adenylate ester analogs include, without limitation, β,γ-methyleneadenosine 5′-triphosphate (AMP-PCP). The antimicrobial activity can be enhanced by high temperature synthesis (e.g., at around 100° C.).

In certain embodiments, the nanocluster has a centered diameter distribution ranging from about 1 nm to about 10 nm, including any diameter within this range such as 0.5 nm, 0.75 nm, 1 nm, 1.25 nm, 1.5 nm, 1.75 nm, 2 nm, 2.25 nm, 2.5 nm, 2.75 nm, 3 nm, 3.25 nm, 3.5 nm, 3.75 nm, 4 nm, 4.5 nm, 5 nm, 5.5 nm, 6 nm, 6.5 nm, 7 nm, 7.5 nm, 8 nm, 8.5 nm, 9 nm, 9.5 nm, or 10 nm. In some embodiments, the nanocluster has a diameter of less than 4 nm. In some embodiments, the nanocluster has a diameter of about 1 nm to about 2 nm.

In certain embodiments, the nanocluster is linked to an internalization sequence, a protein transduction domain, or a cell penetrating peptide.

In another aspect, a composition comprising a nanocluster, described herein, for use in a method of treating an infection is provided. In certain embodiments, the composition further comprises a pharmaceutically acceptable excipient or carrier. In some embodiments, the infection is a bacterial infection such as, but not limited to, a Staphylococcus aureus, Klebsiella pneumoniae, Pseudomonas aeruginosa, or Escherichia coli infection.

In certain embodiments, the composition further comprises an antibiotic. Exemplary antibiotics include, without limitation, fluoroquinolones, aminoglycosides, penicillins, tetacyclines, cephalosporins, macrolides, sulfonamides, carbapenems, ansamycins, carbacephems, carbapenems, lincosamides, monobactams, and oxazolidinones. For example, the antibiotic may include a fluoroquinolone such as ofloxacin, moxifloxacin, ciprofloxacin, gemifloxacin, levofloxacin, or finafloxacin, or a derivative thereof.

In another aspect, a method of treating an infection in a subject is provided, the method comprising administering a therapeutically effective amount of a composition comprising a nanocluster described herein to the subject. In some embodiments, the method further comprises administering a therapeutically effective amount of at least one antibiotic in combination with the composition comprising the nanocluster.

Exemplary antibiotics include, without limitation, fluoroquinolones, aminoglycosides, penicillins, tetacyclines, cephalosporins, macrolides, sulfonamides, carbapenems, ansamycins, carbacephems, carbapenems, lincosamides, monobactams, and oxazolidinones. For example, the antibiotic may include a fluoroquinolone such as ofloxacin or a derivative thereof.

In certain embodiments, the subject has a chronic infection. In some embodiments, the subject has an infection including, without limitation, an ear infection, a cutaneous infection, a lung infection, chronic suppurative otitis media (CSOM), an infection associated with cystic fibrosis, tuberculosis, or an infection in a wound. In some embodiments, the infection is associated with formation of a bacterial biofilm in the subject. In certain embodiments, the infection comprises pathogenic bacteria that are resistant to one or more antibiotics. In some embodiments, the subject has previously been treated for the infection with one or more antibiotics that have not successfully cleared the infection. In another embodiment, the infection is an infection (e.g. Pseudomonas) in a subject who has cystic fibrosis.

In certain embodiments, the treatment eradicates all or most biofilm bacteria and planktonic bacteria. In some embodiments, the treatment eradicates all or most persister cells, which may be, for example, in a biofilm or internalized by a macrophage. In some embodiments, the persister cells that are eradicated by the treatment described herein are multidrug tolerant persister cells. Treatment may eradiate persister cells comprising either Gram-negative or Gram-positive bacteria, including, without limitation, Staphylococcus aureus. Klebsiella pneumoniae, Pseudomonas aeruginosa, or Escherichia coli persister cells.

In certain embodiments, multiple cycles of treatment are administered to the subject. For example, nanoclusters described herein may be administered alone or in combination with an antibiotic either intermittently or according to a daily dosing regimen.

Compositions comprising nanoclusters may be administered by any suitable mode of administration. For example, the composition may be administered intravenously, subcutaneously, by inhalation, or topically. Alternatively, the composition may be administered locally at the site of infected tissue. For example, for an ear infection, the composition comprising nanoclusters may be administered locally into the ear canal.

In another embodiment, a method of eradicating bacteria in a biofilm is provided, the method comprising contacting the biofilm with an effective amount of a composition comprising a nanocluster described herein. In some embodiments, the method further comprises contacting the biofilm with an effective amount of at least one antibiotic. The methods described herein may be used to eradicate bacteria, for example, in a biofilm on a medical device, a personal hygiene article, a toiletry, a cosmetic, a disinfectant, a cleaning solution, or in a water treatment or distribution system.

In another embodiment, a method of eradicating dormant bacteria comprising persister cells is provided, the method comprising contacting the dormant bacteria with an effective amount of a composition comprising a nanocluster described herein. In some embodiments, the method further comprises contacting the dormant bacteria with an effective amount of at least one antibiotic. The dormant bacteria may be present, for example, in a biofilm, in a liquid culture, or on an inanimate surface.

In another embodiment, a method of inhibiting a virulence factor of a bacterium is provided, the method comprising contacting the bacterium with an effective amount of a composition comprising a nanocluster described herein. In some embodiments, the bacterium is selected from the group consisting of Staphylococcus aureus, Klebsiella pneumoniae, Pseudomonas aeruginosa, and Escherichia coli. In some embodiments, the virulence factor is Pseudomonas aeruginosa pyocyanin (PYO).

In another aspect, a kit is provided comprising a nanocluster described herein and instructions for treating a bacterial infection. In some embodiments, the kit further comprises an antibiotic.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. Characterization of gold nanoclusters coated with adenosine triphosphate (AuNC@ATP). (FIG. 1A) Schematics of AuNC@ATP, a picture of the solution of as-synthesized AuNC@ATP, was analyzed using UV-Vis spectroscopy to demonstrate the absence of the plasmon resonance band at 520 nm. (FIG. 1B) Transmission electron microscopy (TEM) image of AuNC@ATP. Magnification and scale bar (5 nm) in the pictures. (FIG. 1C) The particle distribution of AuNC@ATP was measured by TEM.

FIGS. 2A-2B. Exposure to AuNC@ATP leads to the activation of stress that disrupts the outer membrane (OM) and cytoplasmic membrane permeability (CM). First, the stationary phase culture of gram-negative bacteria was exposed to AuNC@ATP and Colistin (positive control). Then the permeability of the OM and IM was assessed by measuring the fluorescence of (FIG. 2A) 8-Anilino-1-naphthalene sulfonic acid (ANS) and (FIG. 2B) propidium iodide (PI), respectively. ANS is a compound that changes fluorescence depending on the polarity of its surrounding environment. In the presence of intact gram-negative bacterial cells in an aqueous environment, ANS is weakly fluorescent. Still, if the OM is disturbed, the ANS can penetrate the nonpolar phospholipid bilayer, resulting in a measurable increase in fluorescence. PI is a membrane-impermeable DNA stain; it can only label bacteria with a compromised CM. Still, if the CM is disturbed, the PI can penetrate the CM and binds to DNA, resulting in a measurable increase in fluorescence.

FIGS. 3A-3B. AuNC@ATP kills gram-negative bacteria in the growth-arrested state without causing bacterial cell lysis. (FIG. 3A) the stationary phase culture of gram-negative bacteria resuspended in phosphate-buffered saline (PBS) and exposed to either AuNC@ATP or Ofloxacin for 4 h. After the treatment, the drugs were removed, and the number of surviving bacteria was assessed by measuring the colony-forming unit per millilitre (CFU/mL). (FIG. 3B) AuNC@ATP-mediated no-lytic cell death of the stationary phase culture of gram-negative bacteria. After the treatment of the stationary phase culture of gram-negative bacteria with PBS, AuNC@ATP (16.8 μM), Ofloxacin (8.3 μM) and Colistin (1.3 mM), the presence of proteins in collected supernatant of each treatment was assessed by using a Pierce BCA Protein Assay kit.

FIGS. 4A-4C. The accumulation of unfolded outer membrane proteins (OMPs) causes AuNC@ATP lethality. (FIGS. 4A and 4B) Suppression on the growth of P. aeruginosa (PA14) and its genetic mutant harbouring a genetic deletion of ClpXP protease (ΔClpXP) incubated with AuNC@ATP at different concentrations. The growth of P. aeruginosa in lysogeny broth (LB) was assessed by measuring the optical density at 600 nm (OD_{600 nm}) (N=3). (FIG. 4C) Schematic showing that AuNC@ATP exert their antibacterial activities mainly by inducing stress that triggers multiple perturbations causing accumulation of toxic unfolded OMPs in the periplasmic space.

FIGS. 5A-5C. Persister cells are more susceptible to AuNC@ATP than metabolically active bacterial cells. (FIG. 5A) Schematic showing the isolation of persister cells from the stationary phase culture of P. aeruginosa (PA14) using Ofloxacin. (FIG. 5B) ATP levels were measured in isolated persister cells and exponentially growing PA14. (FIG. 5C) Persister cells and exponentially growing PA14 were redispersed in phosphate-buffered saline (PBS) with AuNC@ATP (without carbon sources). After the AuNC@ATP treatment, the number of surviving bacteria was assessed by measuring the colony-forming unit per millilitre (CFU/mL). Cells during the AuNC@ATP treatments at different concentrations were plated to generate the dose-response curves (N=3).

FIGS. 6A-6B. P. aeruginosa fails to produce pyocyanin in the presence of a sub-lethal dose of AuNC@ATP. (FIG. 6A) Pyocyanin production by P. aeruginosa (PA14) in lysogeny broth (LB) with AuNC@ATP (N=3). The inset shows the chemical structure of pyocyanin. After centrifugation, the pyocyanin was collected, and the optical density was measured at 520 nm (OD_{520 nm}). The pyocyanin concentration was determined by multiplying the OD₅₂₀ values by 17.072, and the results were expressed in μg/mL. (FIG. 6B) Picture of extracted pyocyanin converted colour to red with HCl.

FIGS. 7A-7C. Bacteria do not develop resistance to AuNC@ATP and prevent sub-lethal antibiotic treatment from inducing resistance. (FIG. 7A) Schematic showing the serial passage experiment. The fold change in minimum inhibitory concentration (MIC) was measured as the ratio between the MIC at passage n/initial MIC. (FIG. 7B) Resistance development of susceptible PAO1 during serial passaging in sub-MIC dosing of Ofloxacin, Tobramacy and AuNC@ATP following 21 passages (1 passage per 24 h). (FIG. 7C) Resistance development of susceptible PAO1 during serial passaging in sub-MIC dosing of Ciprofloxacin in the absence or presence of AuNC@ATP (0.56 μM).

FIGS. 8A-8B. AuNC@ATP prevents the cross-resistance triggers by the sub-lethal fluoroquinolones. (FIG. 8A) Schematic showing the disk diffusion test used to determine the antimicrobial susceptibility profile of PAO1 isolate after 21 passages in media containing subinhibitory concentrations of Ciprofloxacin without (PAO1_{Cp21}) and with AuNC@ATP (PAO1_{Cip21-AuNC@ATP}). (FIG. 8B) Cross-resistance of PAO1_{Cip21} and PAO1_{Cip21-AuNC@ATP} against different antipseudomonal antibiotics. The vertical axis labels indicate the antibiotic tested for cross-tolerance, and the horizontal axis labels indicate the fold change in the inhibition zone compared to the susceptible P. aeruginosa (PAO1 ancestor).

FIGS. 9A-9C. Multiple doses administration of AuNC@ATP is not toxic to mice. Effect of AuNC@ATP on hematology (FIG. 9B) and clinical chemistry parameters (FIG. 9C) at 14 days post-treatment at a dose of 38.19 mg/kg administered intraperitoneally (IP) three times a day for 14 days. The parameters evaluated are listed in FIG. 9A. Group ten mice (five female and five male) were used. Phosphate-buffered saline (PBS) was used as vehicle control.

FIGS. 10A-10D. Quantification of ATP amount per AuNC@ATP. (FIG. 10A) Schematic showing how bioluminescent ATP assays work. (FIG. 10B) Linear correlation of luminescence and ATP concentration. (FIG. 10C) Linear correlation of luminescence and AuNC@ATP concentration. (FIG. 10D)) Linear correlation of ATP concentration and AuNC@ATP concentration.

FIGS. 11A-11B. Cell death mediated by AuNC@ATP occurs without releasing periplasmic proteins and cytosolic components into the supernatant. (FIG. 11A) Schematic showing how bioluminescent ATP assays work. (FIG. 11B) quantification of protein concentration in the supernatant after treatment with AuNC@ATP, Colistine and AuNC@ATP-treated cells exposed to Colistin. The data demonstrate the absence of cell lysis after AuNC@ATP treatment.

FIGS. 12A-12B. Killing by AuNC@ATP does not depend on reactive oxygen species (ROS). (FIG. 12A) Schematic showing how intracellular ROS was determined using the fluorescent probe 2′,7′-dichlorofluorescein diacetate (DCFH-DA). (FIG. 12B) Comparison of the ROS production after treating P. aeruginosa with Ofloxacin and AuNC@ATP. Knowing that killing by bactericidal antibiotics does not depend on ROS, Ofloxacin was used as a comparator. The 1.2 fold-change in ROS production upon treatment with both Ofloxacin and AuNC@ATP demonstrates that AuNC@ATP-mediated cell death is not associated with ROS production.

FIG. 13. ATP is not an anti-persister compound. Representative Petri dish showing regrowth of persister cells following treatment of Ofloxacin-induced persister cells (10⁸ CFU/ml) with ATP (10 mM) and AuNC@ATP (4.2 μM), respectively. The images represent Petri dishes from three independent experiments (n=3) for each condition. We noted that the ATP ligand alone did not show bactericidal activity, confirming that the antimicrobial effect was not derived from the surface ligand. The eradication of persister cells by AuNC@ATP is not determined by the surface ligand density but by the entire AuNC@ATP as a whole entity and compound.

### DETAILED DESCRIPTION OF EMBODIMENTS

Compositions comprising nanoclusters comprising a metallic core conjugated to a nucleotide and methods of using them in treating bacterial infections are provided.

Before the present compositions comprising nanoclusters comprising a metallic core conjugated to a nucleotide and methods of using them in treating bacterial infections are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a bacterial cell” includes a plurality of such bacterial cells and reference to “the nanocluster” includes reference to one or more nanoclusters and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “nanocluster” refers to an organic, inorganic, or hybrid nanocluster having a size of 10 nm or less in length. Nanoclusters may have dimensions of 4 nm or less, including 3 nm or less, or 2 nm or less, or 1 nm or less. In some instances, the nanocluster has dimensions of 2 nm or less. In certain embodiments, the nanocluster has a diameter ranging from about 1 nm to about 10 nm, including any diameter within this range such as 0.5 nm, 0.75 nm, 1 nm, 1.25 nm, 1.5 nm, 1.75 nm, 2 nm, 2.25 nm, 2.5 nm, 2.75 nm, 3 nm, 3.25 nm, 3.5 nm, 3.75 nm, 4 nm, 4.5 nm, 5 nm, 5.5 nm, 6 nm, 6.5 nm, 7 nm, 7.5 nm, 8 nm, 8.5 nm, 9 nm, 9.5 nm, or 10 nm. In some embodiments, the nanocluster has a diameter of about 1 nm to about 2 nm.

“Diameter” as used in reference to a shaped structure (e.g., nanocluster, nanocluster, etc.) refers to a length that is representative of the overall size of the structure. The length may in general be approximated by the diameter of a circle or sphere that circumscribes the structure.

The term “persister cells” refers to cells that have entered a non-growing (i.e., dormant) or extremely slow-growing physiological state that renders them less susceptible or resistant to antimicrobial drugs. Such cells may “persist” after planktonic bacterial cells have been eradicated by the immune system or conventional treatment with an antimicrobial agent. Persister cells are commonly found in biofilms.

As used herein, the term “antimicrobial agent” is interchangeable with the term “antibiotic” and refers to any agent capable of having bactericidal or bacterial static effects on growth. Antibiotics include, but are not limited to, a β-lactam antibiotic, an aminoglycoside, an aminocyclitol, a quinolone, a tetracycline, a macrolide, a lincosamide, a glycopeptide, a lipopeptide, a polypeptide antibiotic, a sulfonamide, trimethoprim, chloramphenicol, isoniazid, a nitroimidazole, a rifampicin, a nitrofuran, methenamine, and mupirocin.

The term “anti-bacterial effect” means the killing of, or inhibition or stoppage of the growth and/or reproduction of bacteria.

The term “efflux pump” as used herein refers to a protein assembly, which transports or exports substrate molecules from the cytoplasm or periplasm of a cell, in an energy-dependent or independent fashion. The term “efflux pump activity” as used herein refers to a mechanism responsible for export of substrate molecules, including antimicrobial agents, outside the cell. The term “efflux pump inhibitor” as used herein refers to a compound, which interferes with the ability of an efflux pump to transport or export a substrate, including antimicrobial agent.

The term “treatment” as used herein refers to (1) the prevention of infection or reinfection (prophylaxis), (2) the eradication of an existing infection, or (3) the reduction or elimination of symptoms of an infectious disease of interest (therapy).

By “therapeutically effective dose or amount” of nanoclusters is intended an amount that, when administered alone or in combination with an antibiotic, as described herein, brings about a positive therapeutic response, such as improved recovery from an infection, including any infection caused by Gram-positive or Gram-negative bacteria. Additionally, a therapeutically effective dose or amount may eradicate persister cells as well as other bacterial cells, including planktonic bacteria as well as bacteria in biofilms, increase ROS accumulation in macrophages, stimulate TNF-α secretion from activated macrophages, restore autophagy, and/or deplete glutathione, catalases, and hydroperoxide reductases. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Substantially purified” generally refers to isolation of a component such as a substance (compound, nanocluster, nucleic acid, polynucleotide, RNA, DNA, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

“Isolated” refers to an entity of interest that is in an environment different from that in which it may naturally occur. “Isolated” is meant to include entities that are within samples that are substantially enriched for the entity of interest and/or in which the entity of interest is partially or substantially purified. By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “recipient”, “individual”, “subject”, “host”, and “patient”, are used interchangeably herein and refer to any vertebrate subject for whom diagnosis, treatment, or therapy is desired, particularly humans. By “vertebrate subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

“Biocompatible” generally refers to a material and any metabolites or degradation products thereof that are generally non-toxic to the recipient and do not cause any significant adverse effects to the subject.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, DC, which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The term “hydrophilic polymer” refers to a material that has the property of dissolving in, absorbing, or mixing easily with water, and comprises repeating units constituting a molecular weight of at least 200 up to 8,000 or more. Hydrophilic polymers include, without limitation, polyethylene glycol (PEG) as well as other materials, which can be used to solubilize nanoclusters. Materials for this purpose include polyethylene glycol (PEG), polyoxyethylene, polymethylene glycol, polytrimethylene glycols, polyvinyl-pyrrolidones, poly lysine (D or L) and derivatives, and polyoxyethylene-polyoxypropylene block polymers and copolymers. The hydrophilic polymers can be linear or multiply branched, and may include multi-arm block copolymers. The hydrophilic polymer renders the nanoclusters soluble when attached thereto in sufficient numbers.

### Nucleotide-Functionalized Nanoclusters for Treatment of Bacterial Infections

Compositions comprising functionalized nanoclusters and methods of using them in treating bacterial infections are provided. In particular, functionalized nanoclusters comprising nucleotides are useful for treating chronic infections associated with production of bacterial biofilms, which are not responsive to conventional antibiotic treatment. Without being bound by theory, bacteria in biofilms tend to be more resistant to treatment with antibiotics, in part, because the biofilm extracellular matrix and outer layers of cells protect bacterial cells in the interior. In addition, many bacterial cells in a biofilm adopt a dormant phenotype, becoming metabolically inactive, which makes them less susceptible to antibiotics that need to be metabolized in order to be effective (e.g., penicillin requires cell wall remodeling in an active bacterial cell in order to cause cell death). Dormant cells in biofilms, which have entered a non-growing or extremely slow-growing physiological state, and as a result have become resistant to antimicrobial drugs, are referred to herein as “persister cells” because of their ability to persist after other active bacterial cells have been eradicated by the immune system and antimicrobial agents. Persister cells are often associated with chronic infections because of the difficulty of eradicating them with conventional antibiotic treatment. The methods described herein are especially useful for treating chronic infections to render persister cells in biofilms more susceptible to antibiotic treatment.

In certain embodiments, a nanocluster is conjugated to a nucleotide such as, but not limited to, adenosine triphosphate (ATP) or a phosphorothioate analog, a deoxyribonucleotide analog, a 7-deaza purine nucleotide analog, or a phosphomethylphosphonic acid adenylate ester thereof. Exemplary, phosphorothioate analogs include, without limitation, ATPαS, ATPβS, or ATPγS. Exemplary deoxyribonucleotide analogs include, without limitation, deoxyadenosine triphosphate (dATP). Exemplary 7-deaza purine nucleotide analogs include, without limitation, 7-deazaadenosine-5′-triphosphate (7-deaza-ATP). Exemplary phosphomethylphosphonic acid adenylate ester analogs include, without limitation, β,γ-methyleneadenosine 5′-triphosphate (AMP-PCP).

The nanocluster is typically spherical in shape, but nanoclusters having other shapes may also be used. For example, the nanocluster may have a shape such as, but not limited to, a sphere, a spheroid (e.g., an oblate or prolate spheroid), an ellipsoid, a rod, a cone, a cube, a cuboid (e.g., a hexahedron), a pyramid, an icosahedron, a truncated icosahedron, or an irregular shape, etc. In certain instances, combinations of different shapes of nanoclusters may be included in a composition. In some embodiments, the nanocluster is substantially spherical in shape, and thus may have dimensions measured as a diameter of a sphere. In certain embodiments, the nanocluster has a centered diameter distribution ranging from about 1 nm to about 10 nm, including any diameter within this range such as 0.5 nm, 0.75 nm, 1 nm, 1.25 nm, 1.5 nm, 1.75 nm, 2 nm, 2.25 nm. 2.5 nm, 2.75 nm, 3 nm, 3.25 nm, 3.5 nm, 3.75 nm, 4 nm, 4.5 nm, 5 nm, 5.5 nm, 6 nm, 6.5 nm, 7 nm, 7.5 nm, 8 nm, 8.5 nm, 9 nm, 9.5 nm, or 10 nm. In some instances, a substantially spherical nanocluster has an average diameter of 2 nm or less. In some embodiments, a substantially spherical nanocluster has an average diameter of about 1 nm to about 2 nm.

The nanocluster may comprise, for example, a metal, a ceramic, carbon-based nanomaterials, silicon or silica, boron, polymers, lipids, or proteins. In certain embodiments, the nanocluster comprises a metallic core conjugated to a nucleotide. The metallic core may comprise a single type of metal atom or more than one type of metal atom, such as two or three, or more different types of metal atoms. In some embodiments, the nanocluster comprises a metal including, without limitation, one or more of gold, silver, platinum, titanium, palladium, rhodium, ruthenium, tin, nickel, copper, aluminum, or an oxide, carbide, nitride, or alloy thereof. In other embodiments, the nanocluster is composed of an oxide of silicon, aluminum, a transition metal (e.g., titanium, zirconium, and the like), aluminosilicate, boron nitride, or a combination thereof. Exemplary materials that may be used in nanoclusters include, but are not limited to, silicon dioxide (e.g., silica), titanium dioxide, silicon-aluminum-oxide, aluminum oxide, and iron oxide. In some instances, the nanocluster is composed of other inorganic materials, such as, but not limited to, diatomaceous earth, calcium hydroxyapatite, and the like. Nanoclusters may also be composed of hydrophobic polymers such as, but not limited to, polylactide; polylactic acid; polyolefins, such as polyethylene, poly(isobutene), poly(isoprene), poly(4-methyl-1-pentene), polypropylene, ethylenepropylene copolymers, and ethylenepropylene-hexadiene copolymers; ethylene-vinyl acetate copolymers; and styrene polymers, such as poly(styrene), poly(2-methylstyrene), styrene-acrylonitrile copolymers, and styrene-2,2,3,3-tetrafluoro-propyl methacrylate copolymers. Nanoclusters may also be composed of natural polymers such as proteins, including, without limitation, albumin, silk, keratin, collagen, elastin, corn zein, and soy protein-based nanoclusters; or polysaccharide-based polymers, including, without limitation, chitosan, hyaluronic acid, alginate, glucan, dextran, and cyclodextrin-based nanoclusters. Carbon-based nanoclusters may include, without limitation, carbon nanotubes, graphite, graphene, fullerenes and nanodiamonds. Combinations of the above materials may also be included in nanoclusters. In certain embodiments, the nanocluster is biocompatible with human cells.

In certain embodiments, the nanocluster is linked to an internalization sequence, a protein transduction domain, or a cell penetrating peptide to facilitate entry into a cell. Cell penetrating peptides that can be used include, but are not limited to, human immunodeficiency virus (HIV)-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1, which can readily transport nanoclusters across plasma membranes (see, e.g., Lai et al. (2023) Bioconjug. Chem. 34 (1): 228-237; Peng et al. (2014) Biomaterials 35 (21): 5605-5618; Jones et al. (2012) J. Control Release 161 (2): 582-591; Fonseca et al. (2009) Adv. Drug Deliv. Rev. 61 (11): 953-64; Schwarze et al. (1999) Science. 285 (5433): 1569-72; Derossi et al. (1996) J. Biol. Chem. 271 (30): 18188-18193; Fuchs et al. (2004) Biochemistry 43 (9): 2438-2444; and Yuan et al. (2002) Cancer Res. 62 (15): 4186-4190).

### Conjugation

Surface functionalization of nanoclusters may be performed by any method known in the art. Functionalization of a nanocluster involves conjugation of a nucleotide (e.g., ATP, dATP, ATPαS, ATPβS, ATPγS, 7-deaza-ATP, or AMP-PCP) to a molecule on the outer surface of the nanocluster. A surface coating may be applied to nanoclusters to introduce functional groups to facilitate attachment of agents. For example, gold nanoclusters with surface coatings comprising thiol, carboxyl, amine, aldehyde, hydroxyl, or azide groups, polyethylene glycol (PEG), dextran, streptavidin, or maleimide and compounds to facilitate bioconjugation are commercially available from a number of companies (e.g., SigmaAldrich (St. Louis, MO), and Cytodiagnostics (Burlington, Ontario, Canada), Creative Diagnostics (Shirley, NY), and Nanocs (New York, NY)). An agent may be conjugated to a nanocluster directly or indirectly through a linker. Exemplary linkers include, without limitation, thioC6 linker (thiohexyl), PEG polymers, diethylenetriaminepentaacetic acid (DTPA), 1,4,7,10-tetraazacyclododecane-1,4,7,10-tetraacetic acid (DOTA), and hydrazide compounds. For a discussion of bioconjugation techniques, see, e.g., Chemistry of Bioconjugates: Synthesis, Characterization, and Biomedical Applications (R. Narain ed., Wiley, 2014), G. T. Hermanson Bioconjugate Techniques (Academic Press. 3^{rd} edition, 2013), and Bioconjugation Protocols: Strategies and Methods (Methods in Molecular Biology, S. S. Mark ed., Humana Press, 2^{nd} edition, 2011), Avvakumova et al. (2014) Trends Biotechnol. 32 (1): 11-20, Couto et al. (2017) Crit Rev Biotechnol. 37 (2): 238-250, Sivaram et al. (2018) Adv. Healthc Mater. 7 (1), van Vught et al. (2014) Comput Struct Biotechnol J. 9: e201402001; Massa et al. (2016) Expert Opin Drug Deliv 13:1-15; Yeh et al. (2015) PLOS One 10 (7): e0129681; Freise et al. (2015) Mol Immunol. 67 (2 Pt A): 142-152; herein incorporated by reference in their entireties.

A variety of conjugation methods and chemistries can be used to conjugate nucleotides or other agents to a nanocluster. Various zero-length, homo-bifunctional, and hetero-bifunctional crosslinking reagents can be used. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate. Woodward's reagent K (2-ethyl-5-phenylisoxazolium-3′-sulfonate), and carbonyldiimidazole. Homo- and hetero-bifunctional reagents generally contain two identical or two non-identical sites, respectively, which may be reactive with amino, sulfhydryl, guanidino, indole, or nonspecific groups.

Suitable amino-reactive groups include, but are not limited to, N-hydroxysuccinimide (NHS) esters, imidoesters, isocyanates, acylhalides, arylazides, p-nitrophenyl esters, aldehydes, and sulfonyl chlorides. Suitable sulfhydryl-reactive groups include, but are not limited to, maleimides, alkyl halides, pyridyl disulfides, and thiophthalimides. In other embodiments, carbodiimides soluble in both water and organic solvent, are used as carboxyl-reactive reagents. These compounds react with free carboxyl groups forming a pseudourea that can then couple to available amines, yielding an amide linkage.

in some embodiments, a nucleotide or other agent is conjugated to a nanocluster using a homobifunctional crosslinker. In some embodiments, the homobifunctional crosslinker is reactive with primary amines. Homobifunctional crosslinkers that are reactive with primary amines include NHS esters, imidoesters, isothiocyanates, isocyanates, acylhalides, arylazides, p-nitrophenyl esters, aldehydes, and sulfonyl chlorides. Non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxycarbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidylpropionate (DSP), and dithiobis(sulfosuccinimidylpropionate (sulfo-DSP). Non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-3′-(dimethylenedioxy)dipropionimidate (DDDP), dimethyl-3,3′-(tetramethylenedioxy)dipropionimidate (DTDP), and dimethyl-3,3′-dithiobispropionimidate (DTBP).

Non-limiting examples of homobifunctional isothiocyanates include: p-phenylenediisothiocyanate (DITC), and 4,4′-diisothiocyano-2,2′-disulfonic acid stilbene (DIDS). Non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate. Non-limiting examples of homobifunctional arylhalides include 1,5-difluoro-2,4-dinitrobenzene (DFDNB), and 4,4′-difluoro-3,3′-dinitrophenyl-sulfone. Non-limiting examples of homobifunctional aliphatic aldehyde reagents include glyoxal, malondialdehyde, and glutaraldehyde. Non-limiting examples of homobifunctional acylating reagents include nitrophenyl esters of dicarboxylic acids. Non-limiting examples of homobifunctional aromatic sulfonyl chlorides include phenol-2,4-disulfonyl chloride, and alpha-naphthol-2,4-disulfonyl chloride. Non-limiting examples of additional amino-reactive homobifunctional reagents include erythritolbiscarbonate, which reacts with amines to give biscarbamates.

In some embodiments, the homobifunctional crosslinker is reactive with free sulfhydryl groups. Homobifunctional crosslinkers reactive with free sulfhydryl groups include, e.g., maleimides, pyridyl disulfides, and alkyl halides. Non-limiting examples of homobifunctional maleimides include bismaleimidohexane (BMH), N,N′-(1,3-phenylene)bismaleimide, N,N′-(1,2-phenylene)bismaleimide, azophenyldimaleimide, and bis(N-maleimidomethyl) ether. Non-limiting examples of homobifunctional pyridyl disulfides include 1,4-di-3′-(2′-pyridyldithio) propionamidobutane (DPDPB). Non-limiting examples of homobifunctional alkyl halides include 2,2′-dicarboxy-4,4′-diiodoacetamidoazobenzene, α, α′-diiodo-p-xylenesulfonic acid, α, α′-dibromo-p-xylenesulfonic acid. N,N′-bis(b-bromoethyl)benzylamine, N,N′-di(bromoacetyfiphenylhydrazine, and 1,2-di(bromoacetyfiamino-3-phenylpropane.

In some embodiments, a nucleotide or other agent is conjugated to a nanocluster using a heterobifunctional reagent. Suitable heterobifunctional reagents include amino-reactive reagents comprising a pyridyl disulfide moiety; amino-reactive reagents comprising a maleimide moiety; amino-reactive reagents comprising an alkyl halide moiety; and amino-reactive reagents comprising an alkyl dihalide moiety.

Non-limiting examples of hetero-bifunctional reagents with a pyridyl disulfide moiety and an amino-reactive NHS ester include N-succinimidyl-3-(2-pyridyldithio) propionate (SPDP), succinimidyl 6-3-(2-pyridyldithio) propionamidohexanoate (LC-SPDP), sulfosuccinimidyl 6-3-(2-pyridyldithio) propionamidohexanoate (sulfo-LCSPDP), 4-succinimidyloxycarbonyl-α-methyl-α-(2-pyridyldithio) toluene (SMPT), and sulfosuccinimidyl 6-α-methyl-xx-(2-pyridyldithio) toluamidohexanoate (sulfo-LC-SMPT).

Non-limiting examples of heterobifunctional reagents comprising a maleimide moiety and an amino-reactive NHS ester include succinimidyl maleimidylacetate (AMAS), succinimidyl 3-maleimidylpropionate (BMPS), N-gamma-maleimidobutyryloxysuccinimide ester (GMBS)N-gamma-maleimidobutyryloxysulfosuccinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethylicyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl) butyrate (SMPB), and sulfosuccinimidyl 4-(p-maleimidophenyl) butyrate (sulfo-SMPB).

Non-limiting examples of heterobifunctional reagents comprising an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino) hexanoylamino) hexanoate (SIAXX), succinimidyl-6-(((4-(iodoacetyl)-amino)methyl)-cyclohexane-1-carbonyl) ami-nohexanoate (SIACX), and succinimidyl-4 ((iodoacetyl)-amino)methylcyclohexane-1-carboxylate (SIAC).

A non-limiting example of a hetero-bifunctional reagent comprising an amino-reactive NHS ester and an alkyl dihalide moiety is N-hydroxysuccinimidyl 2,3-dibromopropionate (SDBP). A non-limiting example of a hetero-bifunctional reagent comprising an alkyl halide moiety and an amino-reactive p-nitrophenyl ester moiety includes p-nitrophenyl iodoacetate (NPIA).

In another example, a 3-ThioC6 linker can be used to functionalize a nucleotide or other agent with a thiol group to facilitate attachment to nanoclusters. For example, the 3-ThioC6 linker can be used to add a thiol group to a nucleotide. The free thiol can be used as a reactive functional group to attach maleimide compounds or for conjugation through disulfide linkages.

An alternative bioconjugation method uses click chemistry. Click chemistry reactions include the Huisgen 1,3-dipolar cycloaddition copper catalyzed reaction (Tornoe et al., 2002, J Organic Chem 67:3057-64), cycloaddition reactions such as Diels-Alder reactions, nucleophilic substitution reactions (especially to small strained rings like epoxy and aziridine compounds), reactions involving formation of urea compounds, and reactions involving carbon-carbon double bonds, such as alkynes in thiol-yne reactions. See, e.g., Kolb et al., 2004, Angew Chem Int Ed 40:3004-31; Evans, 2007, Aust J Chem 60:384-95: Millward et al. (2013) Integr Biol (Camb) 5 (1): 87-95), Lallana et al. (2012) Pharm Res 29 (1): 1-34, Gregoritza et al. (2015) Eur J Pharm Biopharm. 97 (Pt B): 438-453, Musumeci et al. (2015) Curr Med Chem. 22 (17): 2022-2050, Mckay et al. (2014) Chem Biol21 (9): 1075-1101, Ulrich et al. (2014) Chemistry 20 (1): 34-41, Pasini (2013) Molecules 18 (8): 9512-9530, and Wangler et al. (2010) Curr Med Chem. 17 (11): 1092-1116; herein incorporated by reference in their entireties.

### Pharmaceutical Compositions

A functionalized nanocluster conjugated to a nucleotide (e.g., ATP, dATP, ATPαS, ATPβS, ATPγS, 7-deaza-ATP, or AMP-PCP), as described herein, can be formulated into a pharmaceutical composition optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the nanoclusters or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey): sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the nanoclusters (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising nanoclusters are in unit dosage form, meaning an amount of a composition appropriate for a single dose, in a premeasured or pre-packaged form.

The compositions herein may optionally include one or more additional agents, such as antibiotics, adjuvants, immunostimulatory agents, vaccines, and/or other medications used to treat a subject for an infection. Compounded preparations may include nanoclusters and one or more other agents for treating an infection, such as, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones/fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil. Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox, Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin. Advocid), difloxacin (Dicural. Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin (Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N. H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines against bacteria and infectious diseases, including any vaccine comprising bacterial antigenic proteins or attenuated or dead bacteria and, optionally, adjuvants for boosting an immune response against bacteria, such as vaccines against tuberculosis, diphtheria, tetanus, pertussis, Haemophilus influenzae type B, cholera, typhoid, Streptococcus pneumoniae, and the like.

Alternatively, such agents can be contained in a separate composition from the composition comprising the nanoclusters and co-administered concurrently, before, or after the composition comprising the nanoclusters.

### Administration

At least one therapeutically effective cycle of treatment with a composition comprising a functionalized nanocluster conjugated to a nucleotide (e.g., ATP, dATP, ATPαS, ATPβS, ATPγS, 7-deaza-ATP, or AMP-PCP), as described herein, will be administered to a subject for treatment of a bacterial infection. Bacterial infections that can be treated by the methods described herein include bacterial infections caused by Gram negative bacteria such as, but not limited to, Acinetobacter (e.g., Acinetobacter baumannii). Actinobacillus. Bordetella. Brucella, Campylobacter, Cyanobacteria, Enterobacter (e.g., Enterobacter cloacae). Erwinia, Escherichia coli, Franciscella, Helicobacter (Helicobacter pylori), Hemophilus (e.g., Hemophilus influenzae), Klebsiella (e.g., Klebsiella pneumoniae), Legionella (e.g., Legionella pneumophila), Moraxella (e.g., Moraxella catarrhalis), Neisseria (e.g., Neisseria gonorrhoeae, Neisseria meningitidis), Pasteurella, Proteus (e.g., Proteus mirabilis), Pseudomonas (e.g., Pseudomonas aeruginosa), Salmonella (e.g., Salmonella enteritidis, Salmonella typhi), Serratia (e.g., Serratia marcescens), Shigella, Treponema, Vibrio (e.g., Vibrio cholerae), and Yersinia (e.g., Yersinia pestis), as well as Gram positive bacteria such as, but not limited to, Actinobacteria, such as Actinomyces (e.g., Actinomyces israelii), Arthrobacter, Bifidobacterium, Corynebacterium (e.g., Corynebacterium diphtheriae), Frankia, Micrococcus, Micromonospora, Mycobacterium (e.g., Mycobacterium tuberculosis, Mycobacterium leprae), Nocardia, Propionibacterium, and Streptomyces; Firmicutes, such as Bacilli, order Bacillales including Bacillus, Listeria (e.g., Listeria monocytogenes), and Staphylococcus (e.g., Staphylococcus aureus, Staphylococcus epidermidis), Bacilli (e.g., Bacilli anthracis, Bacilli cereus), order Lactobacillus including Enterococcus, Lactobacillus, Lactococcus, Leuconostoc, Pediococcus, and Streptococcus (e.g., Streptococcus pneumoniae, Streptococcus mutans, Streptococcus sanguinis, Streptococcus pyogenes), Clostridia (e.g., Clostridioides difficile. Clostridium perfringens, Clostridium botulinum, Clostridium tetani. Clostridium sordellii), including Acetobacterium, Clostridium, Eubacterium, Heliobacterium, Heliospirillum, Megasphaera, Pectinatus, Selenomonas, Zymophilus, and Sporomusa, Mollicutes, including Mycoplasma (e.g., Mycoplasma pneumoniae), Spiroplasma, Ureaplasma, and Erysipelothrix.

By “therapeutically effective dose or amount” of a nanocluster conjugated to a nucleotide (e.g., ATP, dATP, ATPαS, ATPβS, ATPγS, 7-deaza-ATP, or AMP-PCP) is intended an amount that, when administered alone or in combination with an antibiotic, as described herein, brings about a positive therapeutic response, such as improved recovery from an infection, including any infection caused by Gram-positive or Gram-negative bacteria. Additionally, a therapeutically effective dose or amount may eradicate persister cells as well as other bacterial cells, including planktonic bacteria and bacteria in biofilms. The exact amount required will vary from subject to subject, depending on the species, age, and general condition of the subject, the severity of the condition being treated, the particular type of nanoclusters and their functionalization, other antimicrobial agents or drugs employed in combination, the mode of administration, and the like. An appropriate “effective” amount in any individual case may be determined by one of ordinary skill in the art using routine experimentation, based upon the information provided herein.

In certain embodiments, multiple therapeutically effective doses of compositions comprising nanoclusters, and/or one or more other therapeutic agents, such as antibiotics, adjuvants, immunostimulatory agents, vaccines, and/or other drugs for treating an infection, or other medications will be administered. The compositions comprising nanoclusters are typically, although not necessarily, administered orally, via injection (subcutaneously, intravenously, or intramuscularly), by infusion, topically, or locally. Additional modes of administration are also contemplated, such as intra-arterial, intravascular, pulmonary, intralesional, intraparenchymatous, rectal, transdermal, transmucosal, intrathecal, intraocular, intraperitoneal, and so forth.

The preparations according to the invention are also suitable for local treatment. For example, compositions comprising nanoclusters may be administered directly to the site of infected tissue. The particular preparation and appropriate method of administration can be chosen to target the nanoclusters to sites of chronic infection and sites of bacterial biofilms where persister cells typically reside and require eradication.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising nanoclusters and/or other agents may be administered using the same or different routes of administration in accordance with any medically acceptable method known in the art.

In another embodiment, the pharmaceutical compositions comprising nanoclusters and/or other agents are administered prophylactically, e.g., to prevent infection. Such prophylactic uses will be of particular value for subjects who are immunodeficient, patients who have been treated with immunosuppressive agents, or who have a genetic predisposition or disease (e.g., acquired immunodeficiency syndrome (AIDS), cancer, diabetes, or cystic fibrosis) that makes them prone to developing infections, or subjects in an environment where exposure to infectious bacteria is likely.

In another embodiment, the pharmaceutical compositions comprising nanoclusters and/or antibiotics, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the nanoclusters can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising nanoclusters will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising nanoclusters will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the nanocluster and its type of functionalization, the magnitude of the effect desired, and the route of administration.

Nanoclusters (again, preferably provided as part of a pharmaceutical preparation) can be administered alone or in combination with one or more other therapeutic agents, such as other agents for treating an infection, including, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin: ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones/fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox, Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin (Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N. H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines such as vaccines against tuberculosis, diphtheria, tetanus, pertussis, Haemophilus influenzae type B, cholera, typhoid, and Streptococcus pneumoniae, and other vaccines comprising bacterial antigenic proteins or attenuated or dead bacteria for boosting an immune response against bacteria, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

Nanoclusters can be administered prior to, concurrent with, or subsequent to other agents. If provided at the same time as other agents, nanoclusters can be provided in the same or in a different composition. Thus, nanoclusters and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising nanoclusters and a dose of a pharmaceutical composition comprising at least one other agent, such as another drug for treating an infection, which in combination comprise a therapeutically effective dose, according to a particular dosing regimen. Similarly, nanoclusters and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

### Kits

Kits may comprise one or more containers of the compositions described herein comprising a functionalized nanocluster conjugated to a nucleotide (e.g., ATP, dATP, ATPαS, ATPβS, ATPγS, 7-deaza-ATP, or AMP-PCP), or reagents for preparing such compositions, and optionally one or more antibiotics for treating a bacterial infection. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the functionalized nanoclusters.

In addition to the above components, the subject kits may further include (in certain embodiments) instructions for practicing the subject methods (i.e., instructions for treating a bacterial infection with nanoclusters as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-48 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A nanocluster comprising a metallic core conjugated to a nucleotide, wherein the metallic core has a diameter of less than 10 nm.
2. The nanocluster of aspect 1, wherein the nucleotide is adenosine triphosphate (ATP) or a phosphorothioate analog, a deoxyribonucleotide analog, a 7-deaza purine nucleotide analog, or a phosphomethylphosphonic acid adenylate ester thereof.
3. The nanocluster of aspect 2, wherein the phosphorothioate analog is ATPAS, ATPβS, or ATPyS.
4. The nanocluster of aspect 2, wherein the deoxyribonucleotide analog is deoxyadenosine triphosphate (dATP).
5. The nanocluster of aspect 2, wherein the 7-deaza purine nucleotide analog is 7-deazaadenosine-5′-triphosphate (7-deaza-ATP).
6. The nanocluster of aspect 2, wherein the phosphomethylphosphonic acid adenylate ester is β,γ-methyleneadenosine 5′-triphosphate (AMP-PCP).
7. The nanocluster of any one of aspects 1-6, wherein the diameter of the nanocluster ranges from about 1 nm to about 2 nm as measured using transmission electron microscopy.
8. The nanocluster of aspect any one of aspects 1-7, wherein the metallic core comprises a noble metal.
9. The nanocluster of aspect 8, wherein the metallic core is a gold metallic core.
10. The nanocluster of any one of aspects 1-9, wherein the nanocluster is biocompatible with human cells.
11. The nanocluster of any one of aspects 1-10, wherein the nanocluster is linked to an internalization sequence, a protein transduction domain, or a cell penetrating peptide.
12. A composition comprising the nanocluster of any one of aspects 1-11 for use in a method of treating an infection.
13. The composition of aspect 12, further comprising a pharmaceutically acceptable excipient or carrier.
14. The composition of aspect 12 or 13, further comprising an antibiotic.
15. The composition of any one of aspects 12-14, wherein the infection is a bacterial infection.
16. The composition of aspect 15, wherein the bacterial infection is a Staphylococcus aureus, Klebsiella pneumoniae, Pseudomonas aeruginosa, or Escherichia coli infection.
17. A method of treating an infection in a subject, the method comprising administering a therapeutically effective amount of the composition of any one of aspects 12-16 to the subject.
18. The method of aspect 17, wherein the infection is a bacterial infection.
19. The method of aspect 18, wherein the bacterial infection is a Staphylococcus aureus, Klebsiella pneumoniae, Pseudomonas aeruginosa, or Escherichia coli infection.
20. The method of any one of aspects 17-19, further comprising administering a therapeutically effective amount of at least one antibiotic in combination with the composition.
21. The method of any one of aspects 17-20, wherein the infection is a chronic infection.
22. The method of any one of aspects 17-21, wherein the infection is an ear infection, a cutaneous infection, a lung infection, a catheter-associated urinary tract infection, or a gastrointestinal infection.
23. The method of any one of aspects 17-22, wherein the infection is associated with formation of a bacterial biofilm in the subject.
24. The method of aspect 23, wherein the biofilm is in a chronic wound in the subject.
25. The method of any one of aspects 17-24, wherein the subject has chronic suppurative otitis media (CSOM), cystic fibrosis, or tuberculosis.
26. The method of any one of aspects 17-25, wherein the infection comprises pathogenic bacteria that are resistant to one or more antibiotics.
27. The method of aspect 26, wherein the subject has previously been treated for the infection with one or more antibiotics that have not successfully cleared the infection.
28. The method of any one of aspects 17-27, wherein said treating eradicates all or most biofilm bacteria and planktonic bacteria.
29. The method of any one of aspects 17-28, wherein said treating eradicates all or most persister cells.
30. The method of aspect 29, wherein the persister cells are in a biofilm or are internalized by a macrophage.
31. The method of aspect 29 or 30, wherein the persister cells are multidrug tolerant persister cells.
32. The method of any one of aspects 29-31, wherein the persister cells comprise Gram-negative or Gram-positive bacteria.
33. The method of aspect 32, wherein the persister cells comprise Staphylococcus aureus, Klebsiella pneumoniae, Pseudomonas aeruginosa, or Escherichia coli.
34. The method of any one of aspects 17-33, wherein multiple cycles of treatment are administered to the subject.
35. The method of any one of aspects 17-34, wherein the composition is administered intravenously, subcutaneously, by inhalation, or topically.
36. The method of any one of aspects 17-34, wherein the composition is administered locally at the site of infected tissue.
37. The method of aspect 36, wherein the infection is an ear infection, and the composition is administered locally into the ear canal.
38. The method of any one of aspects 17-37, wherein the infection is a Pseudomonas infection in a subject who has cystic fibrosis.
39. A kit comprising the nanocluster of any one of aspects 1-11 and instructions for treating a bacterial infection.
40. A method of eradicating bacteria in a biofilm, the method comprising contacting the biofilm with an effective amount of the nanocluster of any one of aspects 1-11.
41. The method of aspect 40, further comprising contacting the biofilm with an effective amount of at least one antibiotic.
42. The method of aspect 40 or 41, wherein the biofilm is on a medical device, a personal hygiene article, toiletry, cosmetic, disinfectant, cleaning solution, or in a water treatment or distribution system.
43. A method of eradicating dormant bacteria comprising persister cells, the method comprising contacting the dormant bacteria with an effective amount of the nanocluster of any one of aspects 1-11.
44. The method of aspect 43, further comprising contacting the dormant bacteria with an effective amount of at least one antibiotic.
45. The method of aspect 43 or 44, wherein the dormant bacteria are in a biofilm, in a liquid culture, or on an inanimate surface.
46. A method of inhibiting a virulence factor of a baterium, the method comprising contacting the bacterium with an effective amount of the nanocluster of any one of aspects 1-11.
47. The method of aspect 46, wherein the bacterium is selected from the group consisting of Staphylococcus aureus, Klebsiella pneumoniae, Pseudomonas aeruginosa, and Escherichia coli.
48. The method of aspect 47, wherein the virulence factor is Pseudomonas aeruginosa pyocyanin (PYO).

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Eradication of Bacterial Persister Cells By Leveraging Their Low Metabolic Activity Using Adenosine Triphosphate Coated Gold Nanoclusters

### Introduction

The rise of antibiotic resistance and the declining discovery of new antibiotics has created a global health crisis [1-3]. In addition, we knew that bacteria could survive lethal doses of antimicrobial agents, not through genetic mutations that confer the resistance but instead through reducing their metabolism, resulting in the depletion of intracellular levels of adenosine triphosphate (ATP) [4]. In doing so, they enter a low metabolic state called persister cells, which exhibit multidrug tolerance [5, 6]. Persister cells do not grow in the presence of antimicrobial agents but can resuscitate after the treatment [7, 8]. Therefore, recurrent and chronic infections are generally associated with persister cells [8-12].

Evidence has now accumulated, demonstrating that persister cells promote antibiotic resistance rates [13, 14]. In addition, we now know that bacteria that encounter antibiotics first become tolerant, and then genetic resistance emerges through this tolerance [15, 16]. Because of this link between antibiotic tolerance and resistance and the rise of antibiotic resistance, there is a pressing need to develop treatments to eradicate persister cells. However, killing persister cells is an unmet clinical need because no Food and Drug Administration (FDA) approved antibiotics are effective against persister cells, and there are no potential prospects in the global preclinical bacterial pipeline [17]. Ongoing research into the application of ultrasmall gold nanoclusters (AuNCs have a total diameter of <4 nm) that exhibit antibacterial activity for bacterial infection treatment and diagnosis has demonstrated its advantages as a potential alternative solution for conventional antibiotics in the fight against multidrug-resistant gram-negative bacteria [18-24].

Most antimicrobial AuNCs were discovered in experiments that tested the ability of compounds to inhibit bacterial growth [25-28]. However, they are often ineffective for treating non-growing that are less metabolically active. For instance, previous attempts to use antimicrobial AuNCs to kill bacterial cells with low metabolic activity have failed, with a 97% reduction in antimicrobial efficacy compared to that achieved in bacteria with an active metabolism [28]. This finding demonstrates that existing antimicrobial AuNCs are inadequate to address persister cells with intracellular ATP depletion. Another key translational hurdle is the production of intracellular reactive oxygen species (ROS), the primary mechanism of action leading to the death of bacteria triggered by existing AuNC strategies, which the risk of turning metabolically active cells into persister cells [29, 30].

In support of this idea, numerous independent studies have shown that compounds inducing ROS production in bacteria promote the formation of persister cells. For example, paraquat, a potent inducer of ROS and a widely used herbicide, pushes the metabolically active bacterial cells to become persister cells [31]. Another example is salicylate-induced ROS also causes an increase in persister cell formation [32]. It has also been shown that sublethal antibiotic treatment leads to multidrug resistance through ROS-induced mutagenesis [33]. These results question the appropriateness of the development of antimicrobial AuNCs, relying on ROS production as the primary mechanism of action to combat antibiotic resistance [23, 28, 34, 35]. Therefore, for antimicrobial AuNCs to stay relevant in the clinical battlegrounds to combat antibiotic resistance, it is imperative to eradicate persister cells, one root cause of antibiotic resistance. In addition, designing a new class of AuNCs with new antimicrobial mechanisms of action that are not dependent on ROS is needed.

Current anti persister cell strategies are based on the paradigm of “awakening” them from their low metabolic state before attempting eradication with traditional antibiotics [36]. Therefore, we created a nontraditional antimicrobial chemotherapy strategy based on AuNCs as an adjuvant to eradicate persister cells by conventional antibiotics [37]. However, persister cells formed by multidrug-resistant bacteria can easily survive this therapeutic approach because when the bacteria become resistant to the partner antibiotic, the AuNC/antibiotic combination fails to eradicate persister cells. Herein, we now show that the low metabolic activity of persister cells can be exploited to target them over their metabolically active counterpart with the outcome of complete eradication.

### Results and Discussion

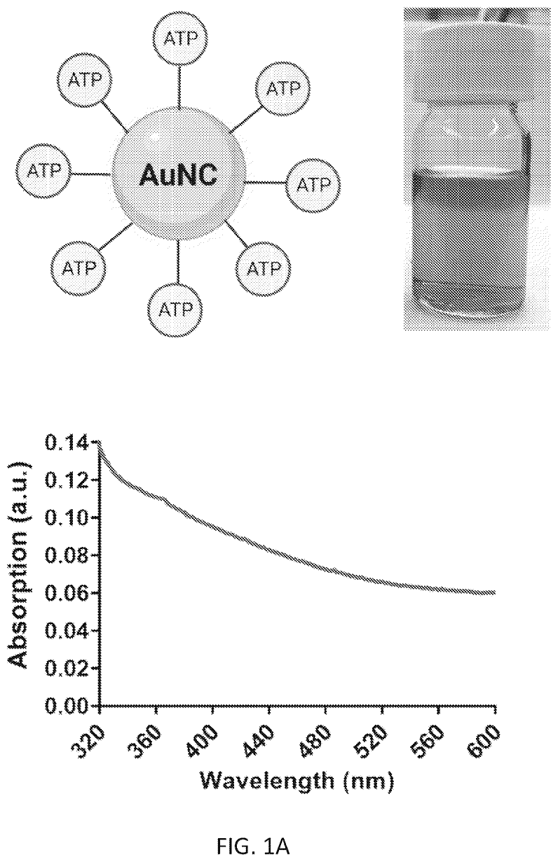

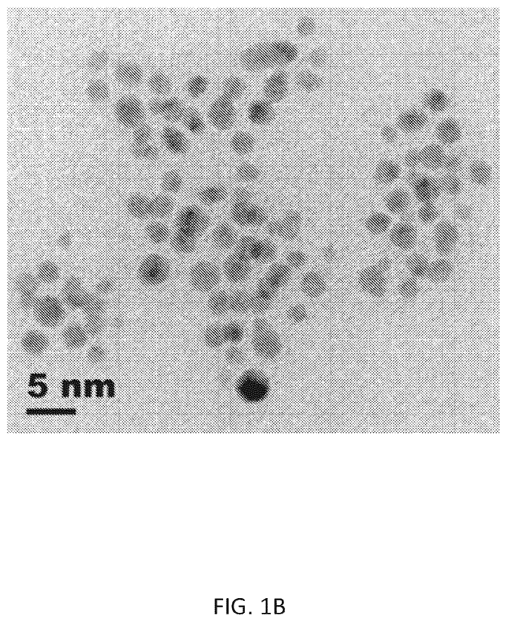

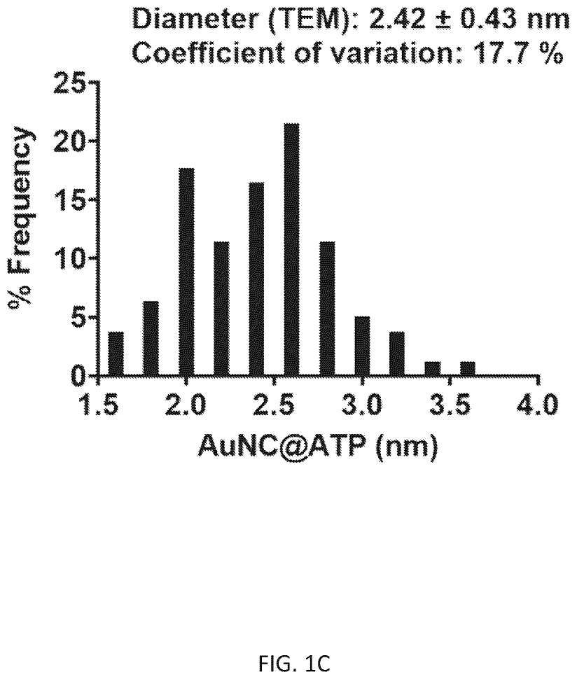

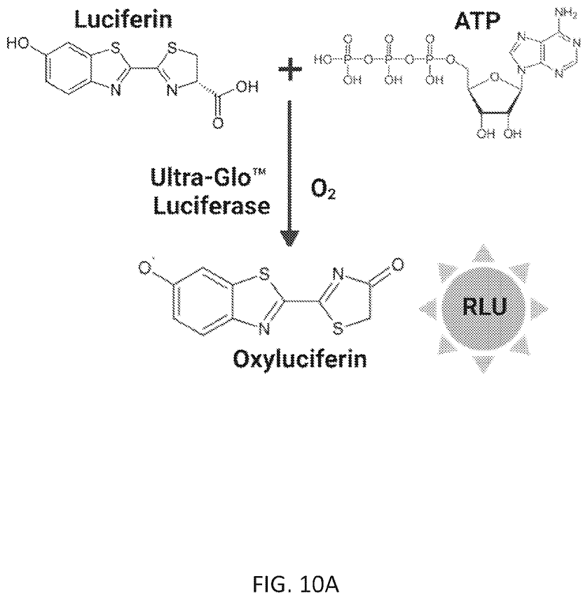

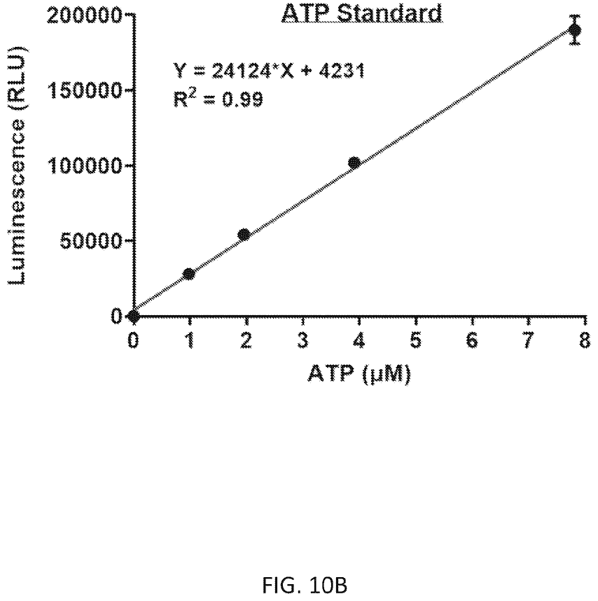

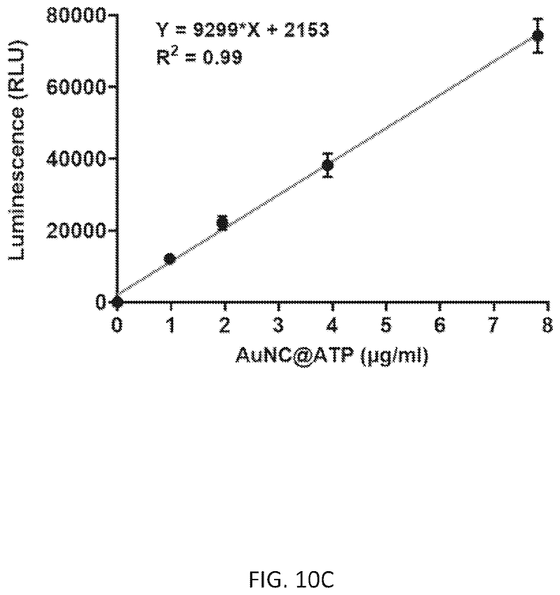

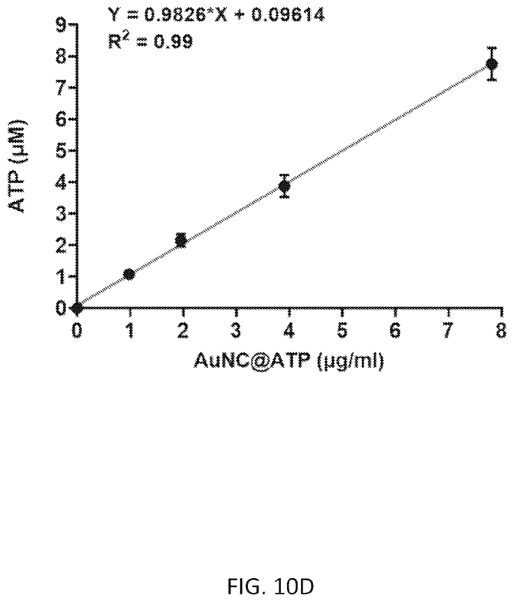

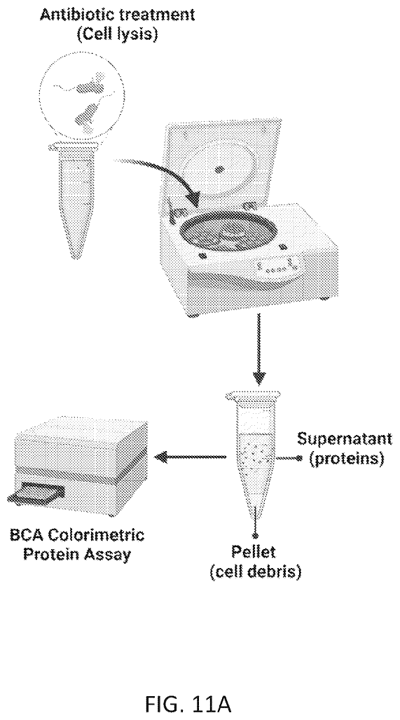

Characterization of gold nanoclusters coated with adenosine triphosphate (AuNC@ATP). FIG. 1 displays the characterization data for the AuNC@ATP. Transmission electron microscopy (TEM) confirms that AuNC@ATP are highly uniform with average core sizes around 2.45±0.43 nm. In addition, the UV-visible spectra show a lack of a prominent surface plasmon peak at around 500 nm, consistent with the small size of the particles [38]. The AuNC@ATP are negatively charged, with a zeta potential in phosphate buffer of −30±2 mV, respectively. The molecular weight (g/mol) of AuNC was calculated as follows: (i) Weight of one AuNC (g)=Volume (nm³)*density of gold (19.32 g/cm³)*10⁻²¹ cm³/nm³=1.487*10⁻¹⁹ g. (ii) Molecular weight (g/mol)=Weight of AuNC (g)*6.022×10²³ mol⁻¹=8.95*104 g/mol. The ATP molecular weight is 507 g/mol, meaning that AuNC (with 2.45 nm in diameter) is 176 times heavier than ATP. Therefore, 99.4% of the weight of AuNC@ATP is due to AuNC. Moreover, the amount of ATP in AuNC@ATP (1 μg/ml or 11.2 nM) was estimated to be 1079 nM based on the ATP bioluminescent assay Kit (FIG. 10). Based on this calculation, we estimate that one AuNC@ATP contains 96 molecules of ATP.

AuNC@ATP disrupts the membrane integrity by increasing permeability without causing bacterial cell lysis. The outer membrane of gram-negative bacteria (OM) is asymmetric, with phospholipids (PLs) in the inner leaflet and lipopolysaccharides (LPS) in the outer leaflet [39]. The biosynthesis of PLs is completed at the cytoplasmic (CM), also known as the inner membrane (IM), which makes the translocation from the IM to the OM (anterograde transport) essential to fill the OM [40]. The translocation of PLs from the OM to the IM (retrograde transport) is believed to maintain the asymmetric LPS/PL structure of OM [40]. In the stationary phase, cells can no longer replace those PLs lost from the IM through new synthesis or stimulating retrograde transport. Thus, the balance between anterograde and retrograde transport is essential for the gram-negative bacteria in the persister cell state to ensure the permeability barrier of OM and integrity of the cytoplasmic membrane required for cell viability [41, 42]. It has previously been observed that anterograde transfer is ATP-independent but could be abolished by ATP hydrolysis [43].

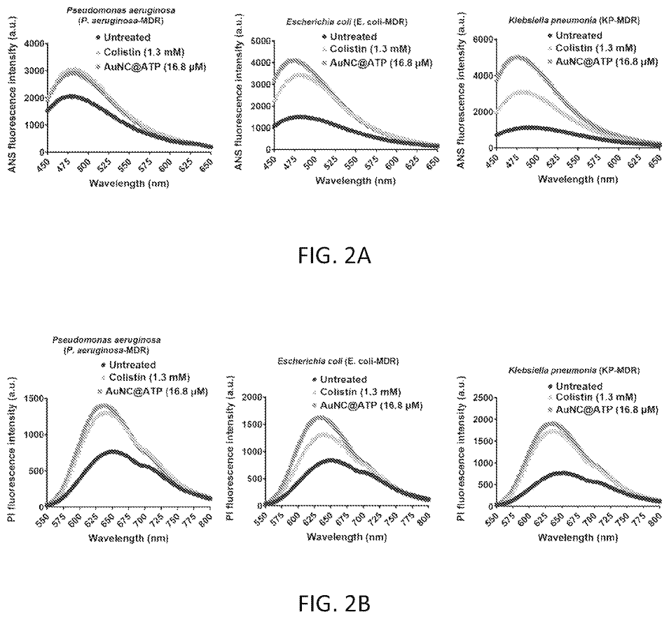

Furthermore, contrary to anterograde transport, ATP drives retrograde PL transport [43]. AuNC@ATP-mediated cell death of the stationary phase occurs through the disruption of lipid homeostasis, which has been shown to activate a novel cell death pathway [44]. We first investigated whether AuNC@ATP-induced alterations of OM permeability using 8-Anilino-1-naphthalene sulfonic acid (ANS), a fluorescent lipophilic dye which shows enhanced fluorescence when in a hydrophobic environment [45-49]. As a positive control, we chose Colistin (polymyxin E) which disrupts the OM [50, 51]. The stationary-phase culture of multidrug-resistant gram-negative bacteria, including Pseudomonas aeruginosa (P. aeruginosa), Escherichia coli (E. coli) and Klebsiella pneumoniae (K. pneumoniae), were treated with either AuNC@ATP or Colistin. We found that the interaction between gram-negative bacteria and AuNC@ATP carries a negative charge on the surface, leading to the impairment of OM permeability, as evidenced by the increased ANS fluorescence (FIG. 2A). For P. aeruginosa, the impact of AuNC@ATP on OM permeability was comparable to the one induced by Colistin. While for E. coli and K. pneumoniae, AuNC@ATP induced more damage than Colistin, which is positively charged (FIG. 2A). Next, we examine whether AuNC@ATP triggers cytoplasmic membrane (CM) disruption. Propidium iodide (PI) is a membrane-impermeant stain which only labels bacteria with a compromised IM [52]. We found that after AuNC@ATP treatment, the PI can penetrate the CM, suggesting damage to CM (FIG. 2B). For P. aeruginosa and K. pneumoniae, the impact of AuNC@ATP on OM permeability was comparable to the one induced by Colistin. While for E. coli, AuNC@ATP induced more damage than Colistin (FIG. 2A).

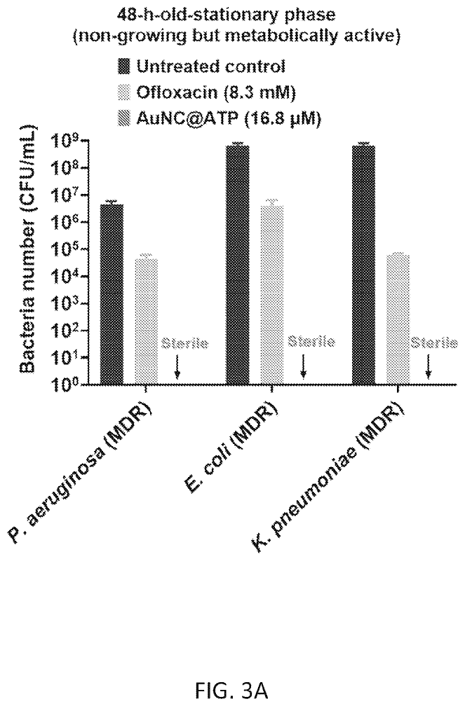

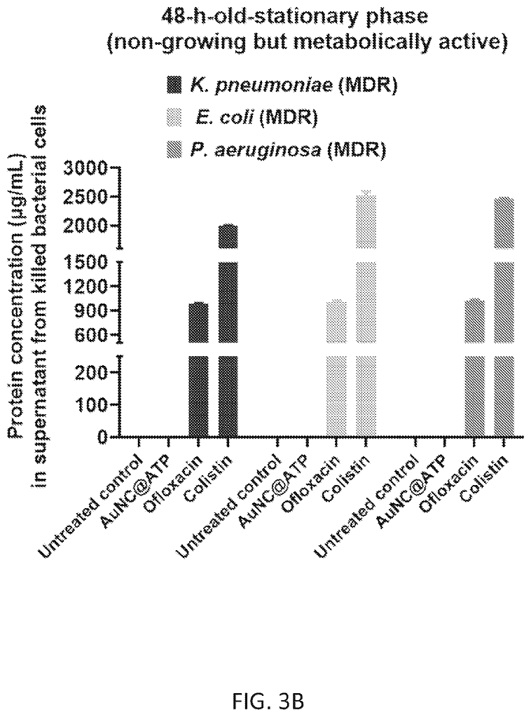

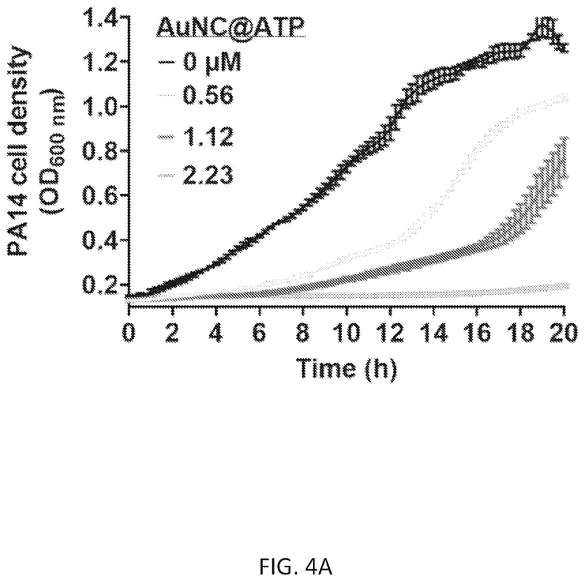

To demonstrate that AuNC@ATP is active against bacteria cells in the growth-arrested (persister cell) phase, we compared the bactericidal activity of AuNC@ATP and Ofloxacin against the stationary-phase gram-negative bacteria. We found that AuNC@ATP sterilizes the stationary-phase culture of P. aeruginosa, E. coli and K. pneumoniae (FIG. 3A). Conversely, Ofloxacin fails to eradicate the stationary-phase culture (FIG. 3A), which broadly supports the work of other studies in this area linking the bacterial metabolic state with antibiotic lethality [4, 53]. Furthermore, these results suggest that exposure to AuNC@ATP triggest a gram-negative cell envelope stress that activates cell death.

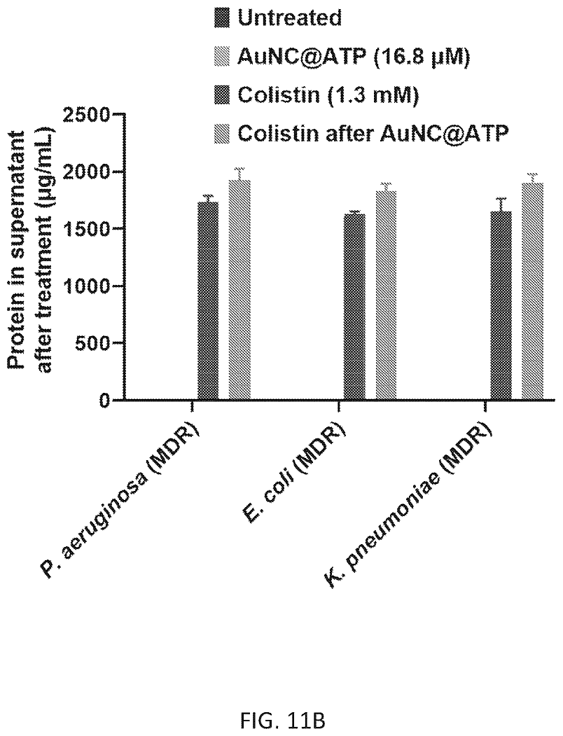

Given that one of the most prominent features of cell death triggered by disruption of PL homeostasis is that cell death does not occur by cell lysis [44]; therefore we analyzed whether AuNC@ATP causes cell lysis using the presence of protein in the supernatant of gram-negative bacteria treated with AuNC@ATP as a proxy of bacterial cell membrane lysis. Colistin and Ofloxacin cause bacteria cell lysis by forming membrane pores and peptidoglycan composition [54-56]. However, in contrast to these conventional antibiotics, no cell lysis was observed after exposure of gram-negative bacteria to AuNC@ATP, as seen by the absence of proteins in the supernatant (FIG. 3B). Moreover, to prove that the entire protein pool was still in bacteria following AuNC@ATP treatment, we lysed the AuNC@ATP-treated bacterial cells with Colistin. As expected, the protein levels were comparable to that obtained from the supernatant of cells treated with Colistin (FIG. 11). Furthermore, AuNC@ATP mediated no lytic cell death, contrary to the mechanism of action of existing antimicrobial AuNCs that results in lysis [28, 57]. This effort showed that AuNC@ATP functions through a mechanism distinct from known classes of bactericidal antibiotics, including cationic antimicrobial polypeptides and existing antimicrobial nanoparticles.

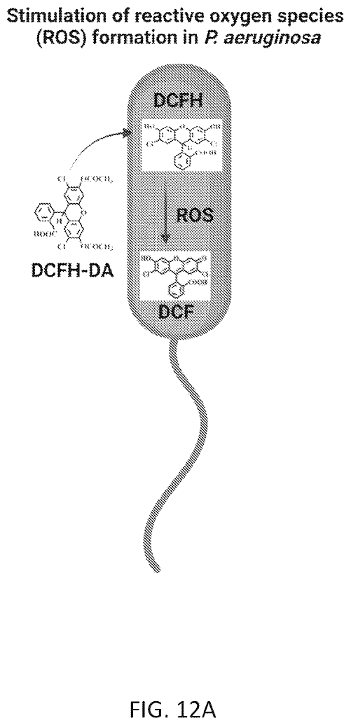

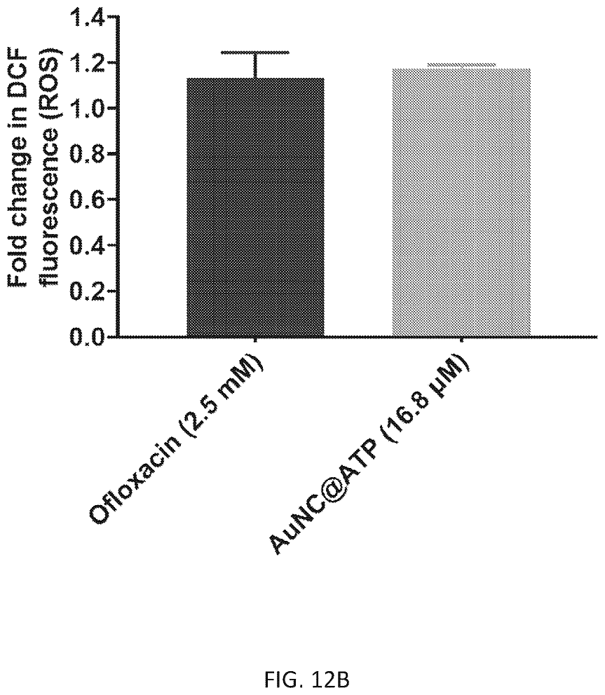

To further support that the mechanism of action of AuNC@ATP is distinct from existing antimicrobial AuNCs, where ROS production is essential to achieve bacterial killing [28, 35], we confirm that the internalization of AuNC@ATP in bacterial cells does not increase intracellular ROS using the fluorescent probe 2′,7′-dichlorofluorescein diacetate (DCFH-DA). As expected, no change in the DCFH-DA fluorescence intensity was observed in the stationary phase culture of P. aeruginosa (PA14) treated with AuNC@ATP compared to Ofloxacin and untreated PA14 cells (FIG. 12). Cumulatively, the data in this section support that after transiting through OM, AuNC@ATP leads to the fusion of the inner leaflet of OM with the outer leaflet of the cytoplasmic membrane, which leads to bacteria death without cell lysis.

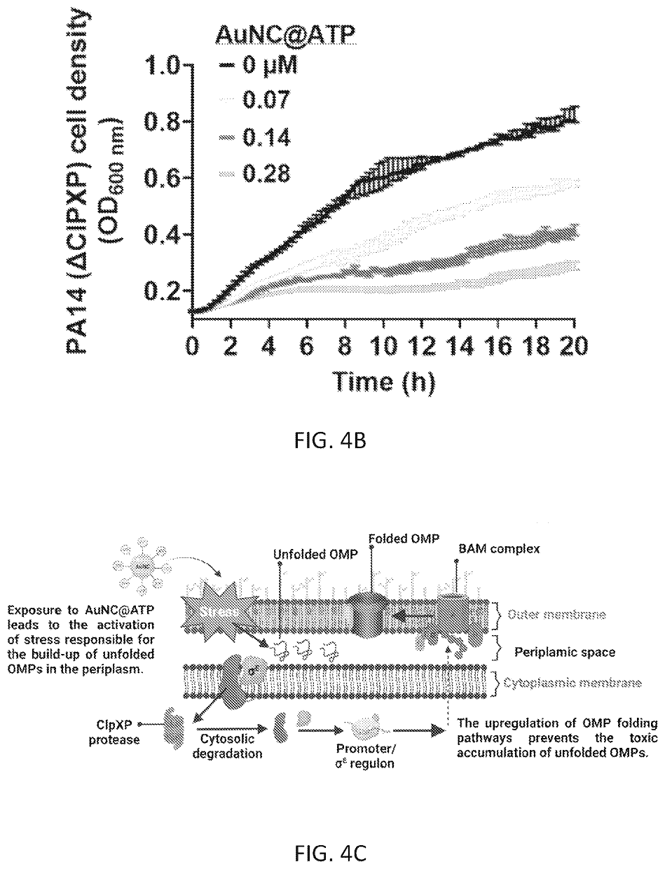

The accumulation of unfolded outer membrane proteins (OMPs) causes AuNC@ATP lethality. We next assessed that ability of AuNC@ATP to induce a stress response that causes a lethal accumulation of unfolded outer membrane proteins (OMPs). Bacteria respond to misfolded or unfolded β-barrel OMPs in the periplasm by inducing sigma factor (σ^{E})-dependent transcription of stress genes in the cytoplasm. The β-barrel structure of OMPs is not found in the cytoplasmic membrane because they represent open pores that enable the diffusion of water, ions and hydrophilic molecules up to 600 Da [58]. To activate the σ^{E} response, unfolded OMPs in the periplasm trigger a proteolytic cascade involving ClpXP, an ATP-dependent cytoplasmic protease that destroys a transmembrane protein (RseA) that usually binds to and inhibits σ^{E} [58, 59]. When the of system is activated, genes are transcribed from σ^{E}-dependent promoters, leading to the upregulation of OMP folding pathways to prevent an accumulation of the highly toxic unfolded OMPs [58]. However, if the σ^{E} response is triggered in cells lacking ClpXP, the upregulation of OMP folding pathways cannot be activated, causing cell death due to the accumulation of the unfolded OMPs in the periplasm. Therefore, to test whether exposure to AuNC@ATP building-up of unfolded OMPs in the periplasm and causing envelope damage, we compared the antimicrobial activity of AuNC@ATP against P. aeruginosa (PA14) and PA14 (ΔClpXP). We found that PA14 (ΔClpXP) is eight times more susceptible to AuNC@ATP than PA14 (ClpXP). The minimum inhibitory concentration (MIC) of PA14 (ClpXP) and PA14 (ΔClpXP) were 2.23 μM and 0.28 μM, respectively (FIGS. 4A and 4B). Cumulatively, this supports that the internalization of AuNC@ATP in bacterial cells induces a lethal accumulation of unfolded OMPs. In line with the fact that cell death mediated by AuNC@ATP occurs without releasing periplasmic proteins and cytosolic components into the supernatant, the hypersusceptibility of PA14 (ΔClpXP) to AuNC@ATP indicates that cell death occurs through the lethal accumulation of unfolded OMPs in the periplasmic space (FIG. 4C and FIG. 11). We conclude that AuNC@ATP has antimicrobial activity attributable to stress responses building up of unfolded OMP accumulation in the periplasm, disrupting bacterial membranes by altering lipid homeostasis and asymmetry.

Although AuNC@ATP-mediated cell death through a lethal accumulation of unfolded OMPs, the molecular mechanism of how it impairs the of response remains unanswered. The GE regulon includes periplasmic chaperones that maintain OMPs in an unfolded state in the periplasm, members of the β-barrel assembly machinery (BAM) complex responsible for inserting OMPs into the OM and periplasmic proteases that degrade misfolded OMPs. If both processes are defective, unfolded OMPs can build up in the periplasm. Further investigation of the open question regarding the molecular mechanism of action will lead to intriguing insights into the potential interaction between AuNC@ATP and the BAM complex or periplasmic proteases. A remarkable feature of the periplasmic space of gram-negative bacteria is that it contains more than 300 proteins and provides a unique protein folding and stabilization environment because it has no ATP [60, 61]. ATP was found to keep the proteins in their soluble form and prevent them from aggregation [62]. Moreover, a proteome-wide profiling analysis suggested that ATP regulates the solubility of a significantly large set of proteins [63]. The computational simulation demonstrates that ATP can unfold a single chain of hydrophobic macromolecules [64]. Thus, these findings partly explain why AuNC@ATP triggers the unfolding of OMPs in the periplasm.

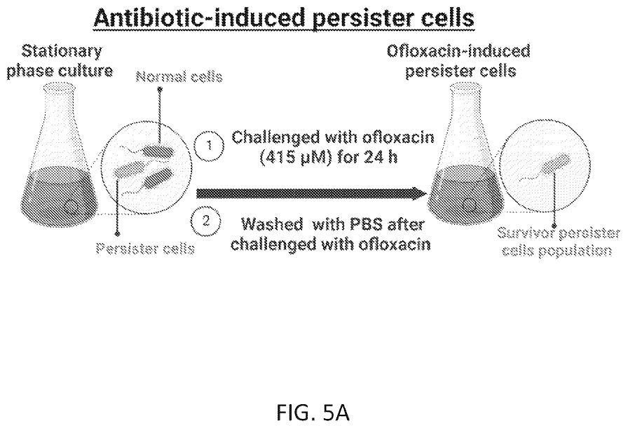

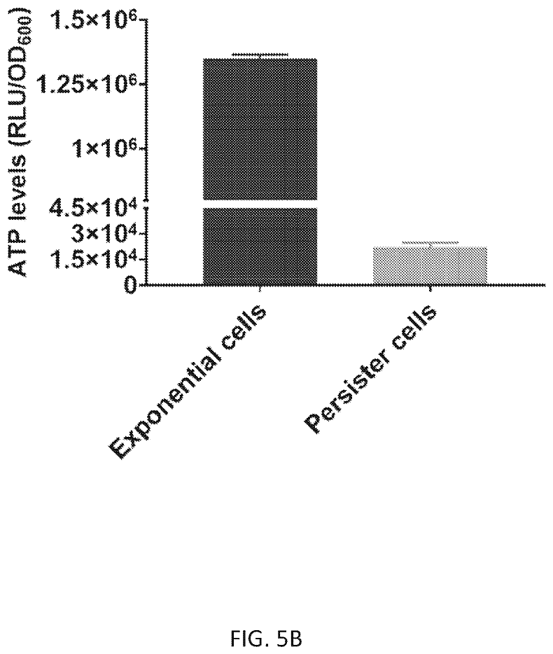

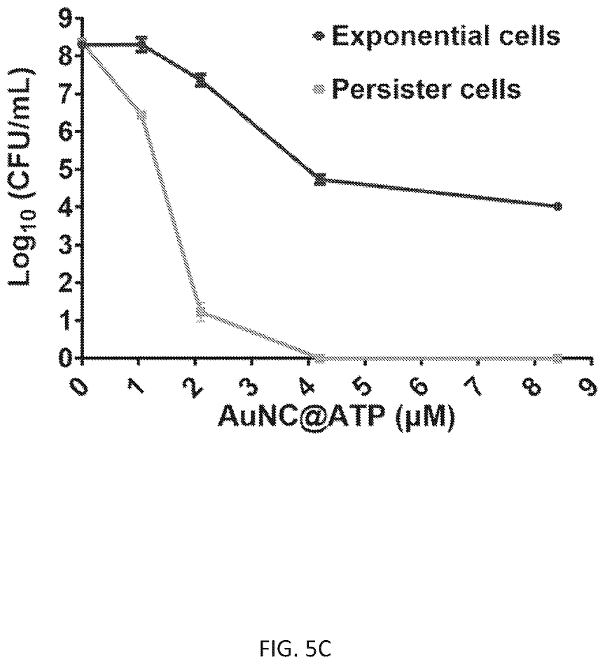

Persister cells are more susceptible to AuNC@ATP than metabolically active bacterial cells. Recent studies have shown a reduction in ATP levels in bacterial persister cells [10, 65, 66]. The decrease in ATP levels correlates with reduced proteolysis of functional proteins by different ATP-dependent proteases [67]. Since AuNC@ATP induces a lethal accumulation of unfolded OMPs, the lethality of AuNC@ATP should increase as bacteria transition from a metabolically active state to a metabolically repressed state (i.e., low ATP levels) due to reduced proteolysis of unfolded OMPs. Moreover, persister cells can no longer synthesize new PLs to replace those lost from OM upon stimulating retrograde transport. We reasoned that these features could be exploited to eradicate persister cells with low metabolic activity. To test this hypothesis, we compared the bactericidal activity of AuNC@ATP against exponential and persister cells. Passage through the stationary phase is associated with the formation of persister cells, which usually represent about 1% of the total bacterial population [9, 12]. Therefore, the 48-h-old-stationary phase was treated with Ofloxacin for 24 h to eradicate non-persister cells (FIG. 5A). Ofloxacin was used at 415 UM, corresponding to 30-fold the MIC for the PAO1 wild-type strain. The surviving persister cells were concentrated and redispersed in phosphate-buffered saline (PBS) to prevent persister cells from awakening from their metabolically repressed state. We confirmed that persister cells have a reduction in ATP levels compared to bacterial cells in the exponential growth phase (FIG. 5B). We found that AuNC@ATP (2.2 μM) leads to a 7-log reduction in bacterial numbers (CFU/ml) compared to less than 1-log reduction when bacteria are in the exponential growth phase (FIG. 5C). Additionally, we sterilized the inoculum of persister cells (10⁸ CFU/ml) with 4.45 UM of AuNC@ATP. However, only a 3-log reduction was observed when bacteria were in the exponential growth phase (from 10⁸ to 10⁵ CFU/ml).

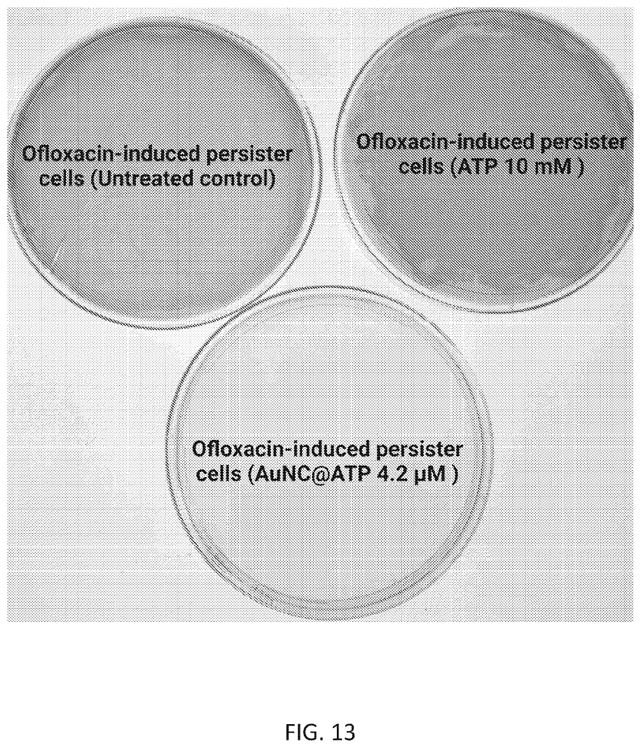

No eradication was observed in persister cells after exposure to the ATP up to 10 mM, proving that the entire entity of AuNC@ATP was required to eradicate persister cells (FIG. 13). Furthermore, contrary to the previous works reporting a 97% reduction in the antimicrobial efficacy of AuNCs when bacteria exhibit low metabolic activity [28], our findings demonstrate that AuNC@ATP-mediated cell death increases as bacteria transition from a metabolically active state to a metabolically repressed state (i.e., low ATP levels). This support the conclusion that AuNC@ATP is a new class of antimicrobial nanoclusters with a unique and novel mechanism of action. This AuNC@ATP-mediated persister cell death is contrary to the bactericidal activity of conventional antibiotics, where lethality decrease as the bacteria transition from a metabolically active state to a metabolically repressed state [4]. This effort showed that contrary to current anti-persister cell strategies are based on the paradigm of “awakening” them from their low metabolic state before attempting eradication with traditional antibiotics, the low metabolic activity of persister cells can be exploited for eradication over their metabolically active counterparts. AuNC@ATP is presented as a benchmark nanocluster that proves the feasibility of this concept.

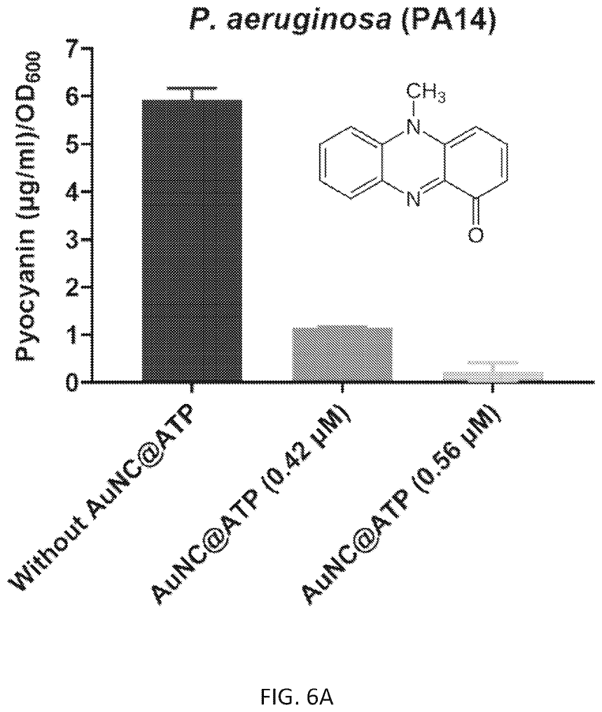

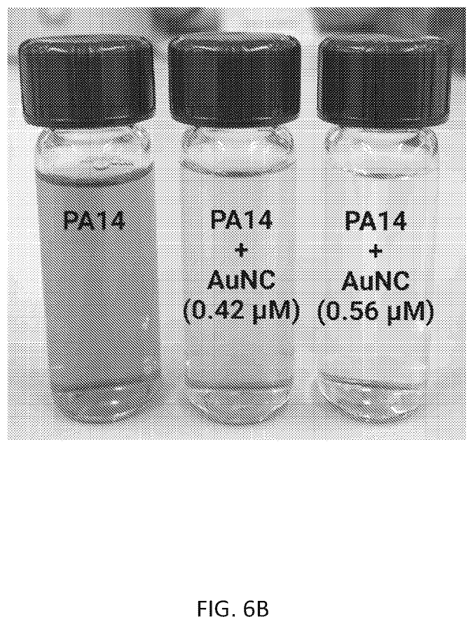

P. aeruginosa fails to produce pyocyanin in the presence of a sub-lethal dose of AuNC@ATP. During infections, P. aeruginosa is often in contact with other pathogens [68]. It has been shown that pyocyanin (PYO), a small molecule produced by P. aeruginosa, increases the persister cell population of other pathogens in contact with P. aeruginosa. The gram-negative coccobacillus Acinetobacter baumannii (A. baumannii) currently leads the WHO list of pathogens in critical need of new therapeutic development. A. baumannii formed 0.07 and 0.02% persister cells in the presence of amikacin and carbenicillin [69]. However, this increased 4- and 3-fold in the presence of PYO [69]. Given that PYO promotes persister cell formation from neighbouring bacteria in coinfection with P. aeruginosa, our next goal was to elucidate whether AuNC@ATP could act as an inhibitor of PYO production. The Chloroform-hydrochloric acid method was used to assess the PYO production. As a result, we found that PYO production by P. aeruginosa (PA14) was 1.15±0.20 μg/mL and 0.23±0.19 μg/mL in the presence of 0.42 and 0.56 UM of AuNC@ATP, respectively, compared to 5.92±0.25 μg/mL in the absence of AuNC@ATP (FIG. 6). We conclude that AuNC@ATP is a multifunctional platform. Therefore, apart from being used to eradicate or prevent the growth of bacteria, AuNC@ATP may act as an anti-virulence agent that can attenuate P. aeruginosa PYO production. Several infections associated with the cytotoxic effects of PYO have been reported [70]. For instance, PYO increases interleukin-8 expression by human airway epithelial cells and mediates tissue damage leading to necrosis during lung infection [72]. The FDA has recently approved five anti-virulence drugs, including two immunoglobulins (BabyBIG and BAT for Clostridium botulinum) and three monoclonal antibodies (raxibacumab and obiltoxaximab for Bacillus anthracis and bezlotoxumab for C. difficile) [73]. Along this line, our data show that although AuNC@ATP is an antimicrobial nanocluster, it could also be used as an anti-virulence drug for P. aeruginosa. In future investigations, it might be possible to use AuNC@ATP for anti-virulence therapy. The rationale is that when virulence traits are suppressed, the bacteria are rendered benign and are more easily cleared by the host's immune system [74].

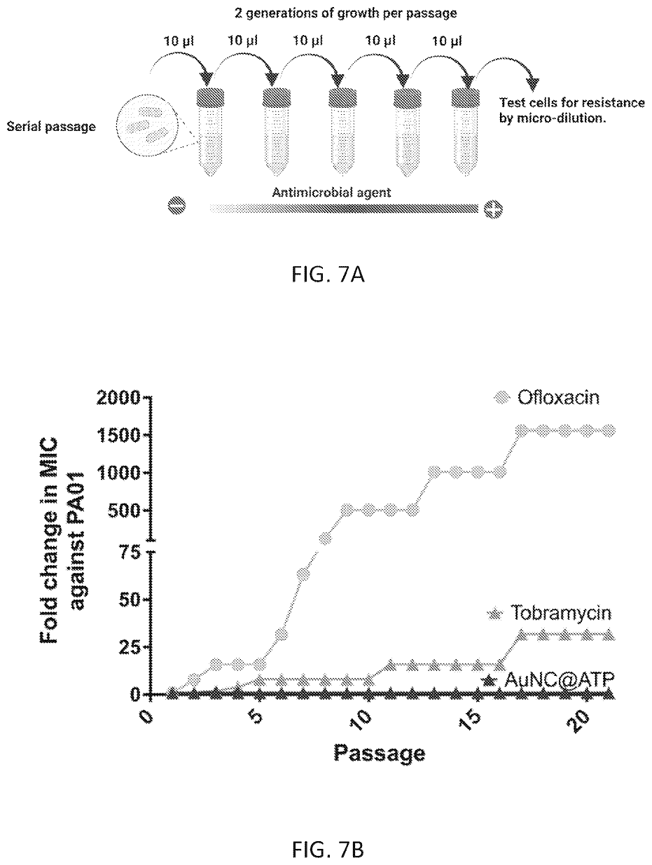

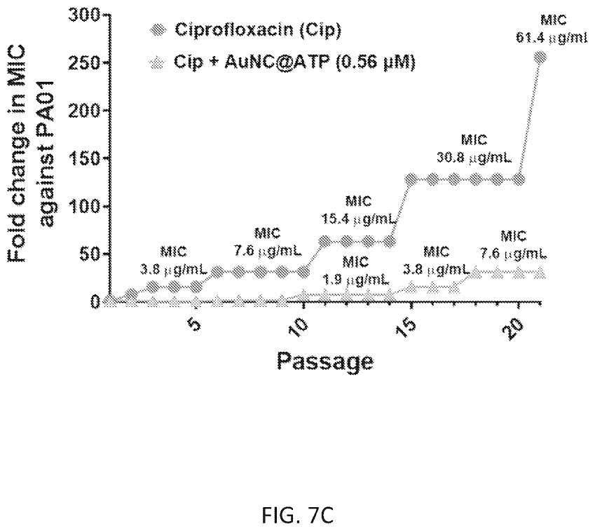

Bacteria do not develop resistance to AuNC@ATP and could prevent sub-lethal antibiotic treatment from inducing multidrug resistance. An ideal antibiotic would show low potential for resistance development alongside an ability to eradicate persister cells. To address resistance rates more quantitatively, we serial-passaged two biologically independent cultures of P. aeruginosa from (PAO1) in sub-lethal concentrations of AuNC@ATP, as well as two control antibiotics: Tobramycin (targeting protein synthesis) and Ofloxacin (targeting DNA gyrase). During 21 passages, we successfully isolated mutants resistant to all the control antibiotics, showing no AuNC@ATP-resistant mutants emerged (FIG. 7A). For Tobramycin and Ofloxacin, the resistance gradually increased throughout the experiment with more than 30-fold and 1500-fold increase in MIC, respectively. While the resistance level remained constant for AuNC@ATP, indicating that P. aeruginosa did not acquire partial resistance to AuNC@ATP. Of note, PAO1 resistance gradually increased after 20 passages in cultivation media containing subinhibitory concentrations of silver nanoparticles [75]. This further supports the conclusion that AuNC@ATP is a new class of antimicrobial nanoclusters with a distinct mechanism of action.

It is suggested that persister cells are the leading cause of the emergence of genetic antimicrobial resistance [14]. Given that AuNC@ATP targets persister cells over their metabolically active counterpart, it could serve as a valuable research tool to kill the multidrug-tolerant subpopulation within an isogenic culture of bacteria genetically susceptible to antibiotics in order to demonstrate that resistance emerges from a multidrug-tolerant subpopulation of bacterial cells. To test this idea, we repeated our serial passaging study with two biologically independent cultures of PAO1. Throughout 21 passages, PAO1 quickly acquires resistance to Ciprofloxacin with a more than 250-fold increase in MIC (FIG. 7B). However, PAO1 resistance to Ciprofloxacin drastically decreases in the presence of AuNC@ATP (1.12 μM), which has been demonstrated earlier in this work to selectively kills persister cells (i.e., >2-log reduction in CFU/ml vs over their metabolically active counterpart) (FIG. 7B). This result supports our conclusion that AuNC@ATP may be a new research tool to demonstrate the association between persister cells and antibiotic resistance development. In addition, this study supports evidence from previous observations that persister cells promote antibiotic resistance rates [14, 66].

AuNC@ATP prevents the cross-resistance that triggers the emergence of superbugs upon exposure to the sub-lethal dose of antibiotics. Superbugs are strains of bacteria that are resistant to several types of antibiotics. Cross-resistance refers to the situation where one antibiotic confers resistance to other drugs within an antibiotic class or to unrelated drugs with different mechanisms of action [76]. Cross-resistance to β-lactam antibiotics is observed in bacterial populations that evolve during exposure of P. aeruginosa to sublethal concentrations of Ciprofloxacin [77]. We demonstrate that a strategy addressing the persister cells is a promising approach to fight against the emergence of multidrug-resistant superbugs. Thanks to the AuNC@ATP, we can now prove that the presence of persister cells within the isogenic culture of bacteria genetically susceptible to Ciprofloxacin is the leading cause of cross-resistance that triggers the emergence of superbugs and their metabolically active counterpart play a minor role. We demonstrated this by examining the antibiotic resistance profile of PAO1 isolate after 21 passages in media containing subinhibitory concentrations of Ciprofloxacin without (i.e., PAO1_{Cip21}) and with AuNC@ATP (i.e., PAO1_{Cip21-AuNC@ATP}).

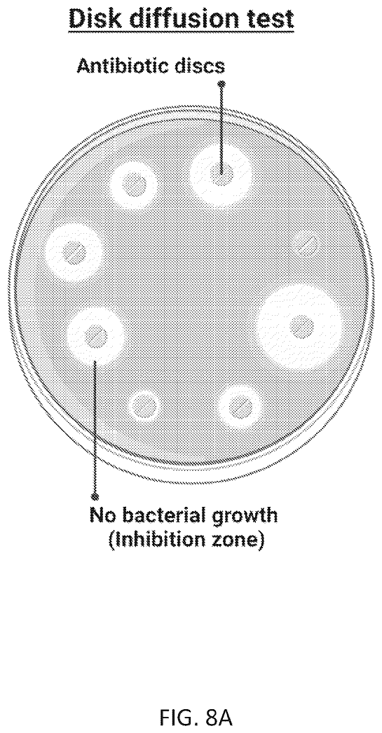

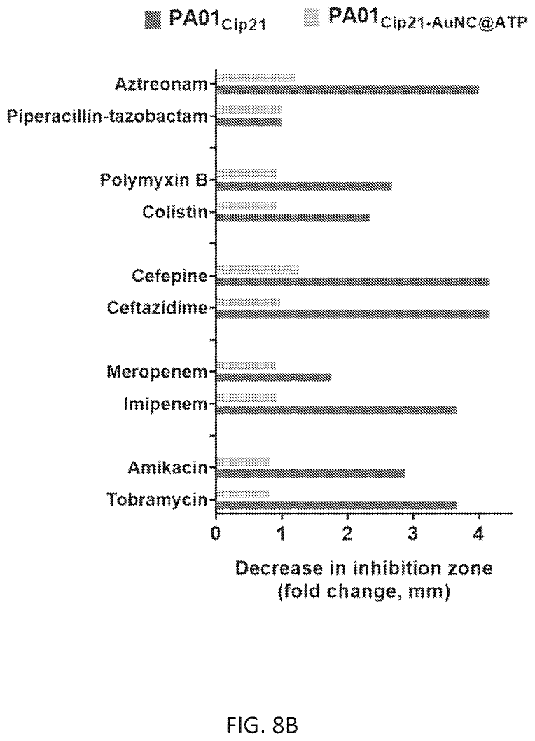

We found that PAO1_{Cip21} is multidrug-resistant to carbapenems, a class of atypical β-lactam antibiotics (Meropenem and Imipenem), aminoglycosides (Tobramycin and Amikacin), polymyxins (Colistin and Polymyxin-B), aztreonam and cephalosporins (Cefepime and Ceftazidime) as evidenced by the decrease in inhibition zone diameter test for antimicrobial activity compared the ancestor PAO1 (FIG. 8). Therefore, it can be assumed that the P. aeruginosa superbug strain (PAO1_{Cp21}) emerges during exposure to sublethal concentrations of Ciprofloxacin. In contrast, PAO1_{Cip21-AuNC@ATP} show an inhibition zone diameter similar to that observed with the ancestor PA01 (FIG. 8). Thus, it can be suggested that persister cells are the leading cause of the emergence of P. aeruginosa superbug strain during exposure to sublethal concentrations of Ciprofloxacin. Furthermore, this study confirms that persister cells are associated with enhanced antibiotic resistance development from fluoroquinolone [78]. Cumulatively, the data in this section lays the groundwork for developing novel nano-antibiotic adjuvants such as AuNC@ATP as it would stop the development of superbugs with the benefit of prolonging the lifespan of current antibiotics.

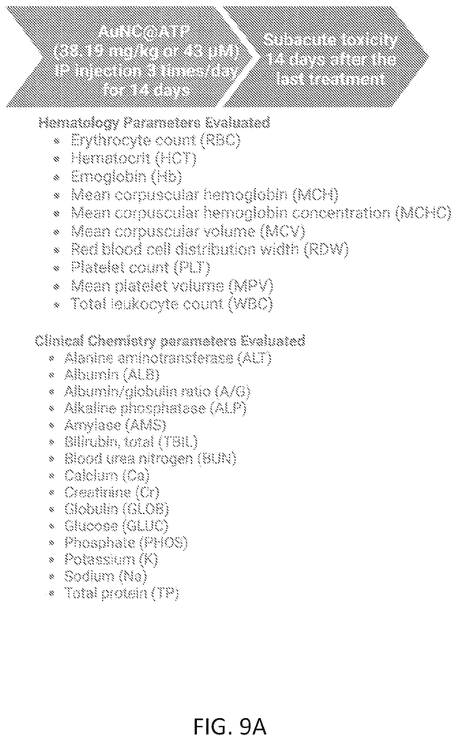

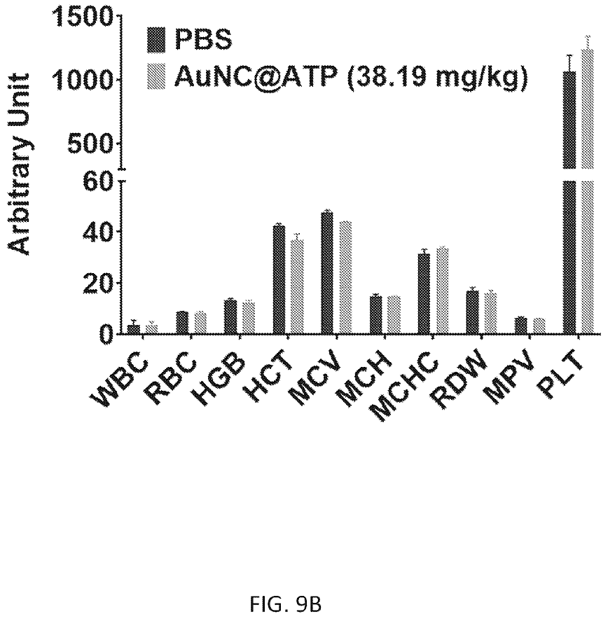

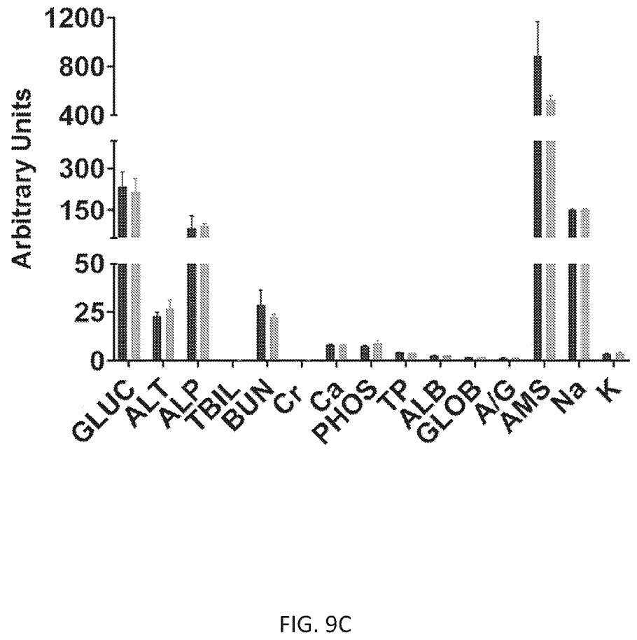

Nonclinical safety and toxicology of AuNC@ATP after intravenous and intraperitoneal injection with multiple doses. The quantitative parameters of toxicity of AuNC@ATP in mice are presented in Table 1. The median lethal dose (LD₅₀) for a single injection of AuNC@ATP administered intravenously (IV) and intraperitoneally (IP) to mice was 205.12 and 346 mg/kg, respectively. For comparison, The LD₅₀ for a single injection of tobramycin sulphate administered by IV and IP to mice was 77 and 262 mg/kg, respectively [79]. Furthermore, the highest tolerated dose of AuNC@ATP that can be administered intravenously to mice without causing any signs of toxicity (i.e., IV-MTD) was 38.19 mg/kg. The IP-MTD was estimated to be between 95 mg/kg (no death) and 195 mg/kg ( 1/10 death mice) for intraperitoneal injection. No clinical laboratory signs of toxicity were found after IP injection of AuNC@ATP 3 times/day for 14 days at a dose equivalent to the IV-MTD. As shown in FIG. 9, we observe that AuNC@ATP did not affect any measured hematology and clinical chemistry parameters. Since AuNCs are cleared from the body through the liver and kidneys [80, 81], one particular interest has been focused on liver and kidney toxicity. Changes in alanine transferase (ALT), aspartate transferase (AST), and total bilirubin (TBIL) levels typically indicate liver injury. The changes in creatinine (CR) and blood urea nitrogen (BUN) levels are associated with kidney injury. No abnormal liver and kidney function-related parameters were observed compared to the control group (PBS) (FIG. 9). Cumulatively, the data in this section demonstrates the lack of toxicity of AuNC@ATP.

### CONCLUSIONS

To summarize, we engineered gold nanoclusters coated with adenosine triphosphate (AuNC@ATP) as a benchmark nanocluster that eradicates persister cells over exponential growth bacterial cells and prove that the low metabolic activity of persister cells can be leveraged for eradication and modulation of pseudomonal virulence. We also demonstrate that AuNC@ATP could serve as a valuable research tool to probe the association between persister cells and the development of antibiotic resistance. Finally, the repeated dose toxicity studies in mice demonstrate its potential safety. Cumulatively, these findings show the promise of AuNC@ATP to eradicate persister cell-driven infectious diseases.

### Experimental Section

### Synthesis of AuNC@ATP

Freshly prepared aqueous solutions of HAuCl₄ (20 mM, 5 mL) and ATP (50 g, 50 mL) were mixed in water (790 mL). After that, an aqueous NaOH solution (1 M, 60 mL) was added to the mixture. Then the mixture was boiled for 5 minutes, and the AuNC@ATP solution was cooled to room temperature. After synthesis, the AuNC@ATP was collected by centrifugation (4000 g for 60 min) of AuNC@ATP in Pall Macrosep Advance centrifugal devices (membrane, MWCO=3000). Finally, the AuNC@ATP was washed three times with deionized water by centrifugation. The resulting AuNC@CPP were lyophilized and dried entirely before further use.

### AuNC@ATP Characterization

Absorption spectra of AuNC@ATP were recorded in the visible domain of the electromagnetic spectrum (400-800 nm) using an absorption spectrophotometer (spectramMax M2, Molecular Devices, Downington, PA). Furthermore, transmission electron microscopy (TEM) images of AuNC@ATP were acquired to analyze the morphology and measure the core size. In addition, the zeta potential of the AuNC@ATP was measured using a Malvern Zetasizer Nano ZS at nanoComposix, San Diago, CA 92111.

### Bacterial Strains, Growth Media, and Conditions

We used multidrug-resistant bacteria from the CDC & FDA Antibiotic Resistance Isolate Bank. In addition, the P. aeruginosa PA01 strain was obtained from ATCC, and the PA14 and PA14 (ΔClpXP) were obtained from Newman's lab at the California Institute of Technology. In all experiments, bacterial cells were cultured in 10 ml of lysogeny broth (LB) at 37° C. and were aerated at 200 rpm in 50 mL plastic polypropylene tubes. Exponential phase cultures were prepared as follows: a stationary overnight culture was diluted 1:1000 in LB and incubated at 37° C. with aeration at 200 rpm until the optical density at 600 nm (OD₆₀₀)=0.3 was reached. The optical density at 600 nm (OD₆₀₀) was read every 15 minutes in a microplate reader (spectramMax M2, Molecular Devices, Downington, PA) to generate growth curves.

### Resistance Development

Serial passage MICs were performed in 96-well microtiter panels. First, an aliquot of the well with the highest concentration permitting growth was taken and back diluted ( 1/100) in new media from inoculated microtiter panels. After overnight incubation at 37° C., this suspension was diluted to a 0.5 McFarland standard turbidity and used to inoculate a new MIC panel, resulting in a final concentration of 1.5×10⁶ CFU/ml. Panels were incubated according to CLSI guidelines, MICs were recorded, and the next inoculum was prepared from the well containing the highest drug concentration that identically allowed growth as described above. Twenty-one repeat passages were performed.

### Susceptibility of Bacteria Using Disc Diffusion Assay

Strains of PA01 (ancestor), PAO1 isolate after 21 passages in media containing subinhibitory concentrations of Ciprofloxacin without (i.e., PAO1_{Cip21}) and with AuNC@ATP (i.e., PAO1_{Cip21-AuNC@ATP}) were tested for susceptibility against a panel of antipseudomonal drugs using the disc diffusion method according to the Clinical Laboratory Standard Institute (CLSI) guidelines. In brief, glycerol stocks of each bacteria were streaked on LB plates and grown overnight at 37° C. An inoculum was prepared by diluting several individual colonies in PBS with a 0.5 McFarland Standard turbidity. The inoculum was then spread with sterile cotton swabs on Mueller Hinton agar plates supplemented with 5% sheep blood. Discs containing 5 μg of antipseudomonal drugs were dispensed on the surface of the plate. After 24 h incubation at 37° C., the inhibition zone was measured using a digital calliper.

### Persister Cells Generation

Persister cells of the P. aeruginosa (PA14) were isolated by treating 250 ml of stationary phase cultures with Ofloxacin (415 UM final concentration). After 24 h treatment, the samples were washed with PBS, and then persister cells were concentred in 10 ml of PBS. The number of persister cells was estimated by serially diluting to determine the colony-forming unit per millilitre (CFU/ml).

### Assessment of Intracellular ATP Level and Total Protein

The ATP levels of exponential and Ofloxacin-induced persisters cells were measured using a BacTiter Glo kit (Promega) according to the manufacturer's instructions. According to the manufacturer's instructions, the protein levels were determined using the bicinchoninic acid (BCA) assay (Thermo Scientific, Pierce).

### Bactericidal Activity Against Persister Cells

All the bactericidal tests were performed on PBS without a carbon source to prevent persister cells from waking up from their low metabolic activity. Ofloxacin-induced persister cells were challenged with antimicrobial agents at the concentrations listed in the text. After the survival bacteria were washed 3× with PBS, the pellet was resuspended in 100 μL of PBS and then spread on an LB plate. The plate was incubated for 72 h at 37° C. before assessing growth.

### In Vivo Cytotoxicity

Animal Treatment and Sample Collection. Stanford University's Administrative Panel approved all animal work in Laboratory Animal Care. The 10-12 week-old BALB/c mice were purchased from Jackson Laboratories (Sacramento, CA) and housed in Stanford University's animal resource facility according to standard guidelines in which food and water were provided ad libitum in a room maintained at 12 h dark/light cycles. Mice (Male=5 each group and Female=5 each group) BALB/c mice were divided into two groups, including control (PBS) and AuNC@ATP. The treatments were administered intravenously (IV) or intraperitoneally (IP) at doses listed in the text. The AuNC@ATP was administered by IP 3 times daily for 14 consecutive days for the sub-acute toxicity study. Then mice were sacrificed 14 days after the last injection. Blood was collected for further investigation of serum chemistry and hematology. Blood samples were subjected to toxicity analysis. An inferior vena cava blood collection was performed at the sacrifice. Blood (150 μL) was placed in a K2 EDTA tube for hematological analysis, and the left blood sample was placed in a 1.5 mL Eppendorf tube for serum extraction. The serum was separated by centrifuging the blood to remove the cellular fraction for liver and renal function testing.

### Generation of Intracellular Reactive Oxygen Species (ROS)

DCFH-DA (2′,7′-dichlorofluorescein diacetate) dye was applied to test the intracellular ROS concentration, which could be cleaved by the intracellular non-specific esterase into the DCFH. DCFH, in the presence of ROS, would be further oxidized into fluorescent DCF (2′,7′-dichlorofluorescein). DCFH-DA in DMSO (10 μM) was added to the treated bacterial solution and further incubated at 37° C., 200 rpm for 15 min. After that, the bacterial cells were centrifuged at 8000 g for 5 min, washed three times with PBS, and resuspended in ultrapure water to the original volume (1 mL). The microplate reader was used to measure the concentration of the produced DCF at the excitation/emission wavelength of 488/525 nm. In this experiment, the fluorescence intensity of DCF directly reflects the amount of ROS generation. The ROS amount was then normalized to the total cell number, which was reflected by the optical density at 600 nm (OD₆₀₀). Finally, the relative ROS production level was calculated by normalizing the ROS level from the treated groups with the production level of the PBS-treated group.

### Outer Membrane Permeability Assay

After treatment with antimicrobial agents, the stationary phase culture of gram-negative bacteria was diluted with PBS to form a suspension with the optical density at 600 nm (OD₆₀₀)=0.5 and add 50 μl of 3 mg/ml 8-Anilino-1-naphthalene sulfonic acid (ANS). After equilibration for 30 min at 37° C., the cells were washed with PBS by centrifugation (8000 g for 5 min) and resuspended in fresh PBS solution (1 ml). Then the fluorescence intensity between 450-650 nm was measured with excitation at 380 nm.

### Cytoplasmic Membrane Permeability Assay

After treatment with antimicrobial agents, the stationary phase culture of gram-negative bacteria was diluted with PBS to form a suspension with the optical density at 600 nm (OD₆₀₀)=0.5 and add 5 μl of 1 mg/ml propidium iodide (PI dissolved in sterile deionized H₂O). After equilibration for 30 min at 37° C., the cells were washed with PBS by centrifugation (8000 g for 5 min) and resuspended in fresh PBS solution (1 ml). Then the fluorescence intensity between 550-800 nm was measured with excitation at 530 nm.

### Pyocyanin Quantitation Assay

Pyocyanin concentration was determined as described by Essar et al. [82]. First, an aliquot of the exponential growth culture of PA14 at the optical density at 600 nm (OD₆₀₀)=0.5 was taken and back diluted ( 1/100) in 50 mL of lysogeny broth (LB) in the absence and presence of sub-lethal doses of AuNC@ATP, incubated at 37° C. and were aerated at 200 rpm to maximize pyocyanin production. After 24 h, the bacterial cells were removed from the cultures by centrifugation (8000 g for 5 min). Then, pyocyanin was extracted from the supernatant by adding Chloroform to a total of 50% total volume. The mixture was vortexed vigorously for 30 seconds, and let the sample settled for 10 minutes to allow for the aqueous phase to separate. Next, the blueish Chloroform fraction was carefully transferred to the new tube. Then 0.1 N HCl was added to 20% total volume and vortexed vigorously for 30 seconds. After this step, the chloroform fraction turns from blue to clear, and the small aqueous fraction of added acid turns from clear to pink. Again, the sample settled for 10 minutes to allow for the aqueous phase to separate. Finally, the aqueous fraction was removed, and the absorbance at 520 nm (OD₅₂₀) was measured. Concentrations, expressed as micrograms of pyocyanin produced per millilitre of culture supernatant, were determined by multiplying the OD₅₂₀ by 17.072. Next, the concentration values were normalized to the cell density of each sample (OD₆₀₀).

### Statistical Analysis

Data are represented as the mean±s.d. Statistical analyses (details in figure legends) were calculated with GraphPad Prism Ver. 9 (GraphPad, San Diego, CA). A p-value of <0.05 was considered statistically significant.

TABLE 1
Values of the median lethal dose and highest tolerated dose
for a single administration of AuNC@ATP to adult mice.

| | Single dose injection of AuNC@ATP | | |
|---|---|---|---|
| | Median lethal | Highest tolerated | |
| Route of administration | dose (LD₅₀) | dose (MTD) | |
| Intravenous injection (IV) | 205.12 mg/kg | 38.19 | mg/kg |
| Intraperitoneal injection (IP) | 345.67 mg/kg | 95 to 195 | mg/kg |

### REFERENCES

[1] C. J. Murray, K. S. Ikuta, F. Sharara, L. Swetschinski, G. R. Aguilar, A. Gray, C. Han, C. Bisignano, P. Rao, E. Wool, The Lancet, 399 (2022) 629-655.
[2] D. Larsson, C. F. Flach, Nature Reviews Microbiology, 20 (2022) 257-269.
[3] S. Bombaywala, A. Mandpe, S. Paliya, S. Kumar, Environmental Science and Pollution Research, 28 (2021) 24889-24916.
[4] A. J. Lopatkin, J. M. Stokes, E. J. Zheng, J. H. Yang, M. K. Takahashi, L. You, J. J. Collins, Nature microbiology, 4 (2019) 2109-2117.
[5] N. R. Cohen, M. A. Lobritz, J. J. Collins, Cell host & microbe, 13 (2013) 632-642.
[6] A. Brauner, O. Fridman, O. Gefen, N. Q. Balaban, Nature Reviews Microbiology, 14 (2016) 320-330.
[7] I. Keren, N. Kaldalu, A. Spoering, Y. Wang, K. Lewis, FEMS microbiology letters, 230 (2004) 13-18.
[8] R. A. Fisher, B. Gollan, S. Helaine, Nature Reviews Microbiology, 15 (2017) 453-464.
[9] K. Lewis, Nature Reviews Microbiology, 5 (2007) 48-56.
[10] K. Lewis, Annual review of microbiology, 64 (2010) 357-372.
[11] P. L. Santa Maria, A. C. Kaufman, B. Bacacao, A. Thai, X. Chen, A. Xia, Z. Cao, A. Fouad, L. A. Bekale, Otology & Neurotology, 42 (2021) e1263-e1272.
[12] K. Lewis, Bacterial biofilms, (2008) 107-131.
[13] N. Q. Balaban, J. Merrin, R. Chait, L. Kowalik, S. Leibler, Science, 305 (2004) 1622-1625.
[14] E. M. Windels, J. E. Michiels, M. Fauvart, T. Wenseleers, B. Van den Bergh, J. Michiels, The ISME journal, 13 (2019) 1239-1251.
[15] K. Lewis, Y. Shan, Science, 355 (2017) 796-796.
[16] I. Levin-Reisman, I. Ronin, O. Gefen, I. Braniss, N. Shoresh, N. Q. Balaban, Science, 355 (2017) 826-830.
[17] U. Theuretzbacher, K. Outterson, A. Engel, A. Karlen, Nature Reviews Microbiology, 18 (2020) 275-285.
[18] K. Zheng, J. Xie, ACS nano, 14 (2020) 11533-11541.
[19] Y. Xie, Q. Zhang, W. Zheng, X. Jiang, ACS Applied Materials & Interfaces, 13 (2021) 35306-35314.
[20] X. Zhao, H. Tang, X. Jiang, ACS nano, 16 (2022) 10066-10087.
[21] W. Zheng, Y. Jia, Y. Zhao, J. Zhang, Y. Xie, L. Wang, X. Zhao, X. Liu, R. Tang, W. Chen, Nano Letters, 21 (2021) 1992-2000.
[22] J. Li, R. Cha, X. Zhao, H. Guo, H. Luo, M. Wang, F. Zhou, X. Jiang, Acs Nano, 13 (2019) 5002-5014.
[23] K. Zheng, M. I. Setyawati, D. T. Leong, J. Xie, Bioactive materials, 6 (2021) 941-950.
[24] Y. Zheng, W. Liu, Z. Qin, Y. Chen, H. Jiang, X. Wang, Bioconjugate Chemistry, 29 (2018) 3094-3103.
[25] M. Tang, J. Zhang, C. Yang, Y. Zheng, H. Jiang, Frontiers in Chemistry, 8 (2020) 181.
[26] S. Yougbare, T. K. Chang, S. H. Tan, J. C. Kuo, P. H. Hsu, C. Y. Su, T. R. Kuo, International journal of molecular sciences, 20 (2019) 2924.
[27] Z. Pang, Q. Li, Y. Jia, W. Yan, J. Qi, Y. Guo, F. Hu, D. Zhou, X. Jiang, Chemical science, 12 (2021) 14871-14882.
[28] K. Zheng, M. I. Setyawati, D. T. Leong, J. Xie, ACS nano, 11 (2017) 6904-6910.
[29] Y. Zheng, M. Wei, H. Wu, F. Li, D. Ling, Journal of Nanobiotechnology, 20 (2022) 1-19.
[30] K. Zheng, J. Xie, Accounts of Materials Research, 2 (2021) 1104-1116.
[31] Y. Wu, M. Vulić, I. Keren, K. Lewis, Antimicrobial agents and chemotherapy, 56 (2012) 4922-4926.
[32] T. Wang, I. El Meouche, M. J. Dunlop, Scientific reports, 7 (2017) 1-7.
[33] M. A. Kohanski, M. A. DePristo, J. J. Collins, Molecular cell, 37 (2010) 311-320.
[34] J. Meng, Y. Gao, W. Li, J. Wang, X. Chen, Talanta, 234 (2021) 122618.
[35] K. Zheng, M. I. Setyawati, D. T. Leong, J. Xie, Chemistry of Materials. 30 (2018) 2800-2808.
[36] K. R. Allison, M. P. Brynildsen, J. J. Collins, Nature, 473 (2011) 216-220.
[37] Z. Cao, X. Chen, J. Chen, A. Xia, B. Bacacao, J. Tran, D. Sharma, L. A. Bekale, P. L. Santa Maria, Nanoscale, 14 (2022) 10016-10032.
[38] V. Amendola, R. Pilot, M. Frasconi, O. M. Maragò, M. A. latì, Journal of Physics: Condensed Matter, 29 (2017) 203002.
[39] H. Nikaido, Microbiology and molecular biology reviews, 67 (2003) 593-656.
[40] E. Lundstedt, D. Kahne, N. Ruiz, Chemical Reviews, 121 (2020) 5098-5123.
[41] R. L. Guest, S. T. Rutherford, T. J. Silhavy, Trends in Microbiology, 29 (2021) 334-345.
[42] J. C. Malinverni, T. J. Silhavy, Proceedings of the National Academy of Sciences, 106 (2009) 8009-8014.
[43] W. Y. Low, S. Thong, S. S. Chng, Proceedings of the National Academy of Sciences, 118 (2021) e2110055118.
[44] H. A. Sutterlin, H. Shi, K. L. May, A. Miguel, S. Khare, K. C. Huang, T. J. Silhavy, Proceedings of the National Academy of Sciences, 113 (2016) E1565-E1574.
[45] L. Stryer, Journal of molecular biology, 13 (1965) 482-495.
[46] D. H. Haynes, H. Staerk, The Journal of Membrane Biology, 17 (1974) 313-340.
[47] K. Young, L. L. Silver, Journal of Bacteriology, 173 (1991) 3609-3614.
[48] F. A. Falchi, E. A. Maccagni, S. Puccio, C. Peano, C. De Castro, A. Palmigiano, D. Garozzo, A. M. Martorana, A. Polissi, G. Deho, Journal of bacteriology, 200 (2018) e00487-00417.
[49] G. Malojčić, D. Andres, M. Grabowicz, A. H. George, N. Ruiz, T. J. Silhavy, D. Kahne, Proceedings of the National Academy of Sciences, 111 (2014) 9467-9472.
[50] A. Sabnis, K. L. Hagart, A. Klöckner, M. Becce, L. E. Evans, R. C. D. Furniss, D. A. Mavridou, R. Murphy, M. M. Stevens, J. C. Davies, Elife, 10 (2021) e65836.
[51] S. Manioglu, S. M. Modaresi, N. Ritzmann, J. Thoma, S. A. Overall, A. Harms, G. Upert, A. Luther, A. B. Barnes, D. Obrecht, Nature communications, 13 (2022) 1-12.
[52] B. Ma, C. Fang, J. Zhang, M. Wang, X. Luo, Z. Hou, Bio-protocol, 10 (2020) e3548-e3548.
[53] K. G. Völzing, M. P. Brynildsen, MBio, 6 (2015) e00731-00715.
[54] A. Khondker, M. C. Rheinstädter, Communications Biology, 3 (2020) 1-4.
[55] S. Vincent, B. Glauner, L. Gutmann, Antimicrobial agents and chemotherapy, 35 (1991) 1381-1385.
[56] M. A. Kohanski, D. J. Dwyer, J. J. Collins, Nature Reviews Microbiology, 8 (2010) 423-435.
[57] L. Wang, S. Li, J. Yin, J. Yang, Q. Li, W. Zheng. S. Liu, X. Jiang, Nano Letters, 20 (2020) 5036-5042.
[58] A. M. Mitchell, T. J. Silhavy, Nature Reviews Microbiology, 17 (2019) 417-428.
[59] A. Konovalova, M. Grabowicz, C. J. Balibar, J. C. Malinverni, R. E. Painter, D. Riley, P. A. Mann, H. Wang, C. G. Garlisi, B. Sherborne, Proceedings of the National Academy of Sciences, 115 (2018) E6614-E6621.
[60] W. J. Allen, G. Phan, G. Waksman, Advances in Protein Chemistry and Structural Biology, 78 (2009) 51-97.
[61] J. H. Weiner, L. Li, Biochimica et Biophysica Acta (BBA)-Biomembranes, 1778 (2008) 1698-1713.
[62] A. Patel, L. Malinovska, S. Saha, J. Wang, S. Alberti, Y. Krishnan, A. A. Hyman, Science, 356 (2017) 753-756.
[63] S. Sridharan, N. Kurzawa, T. Werner, I. Günthner, D. Helm, W. Huber, M. Bantscheff, M. M. Savitski, Nature communications, 10 (2019) 1-13.
[64] S. Sarkar, J. Mondal, The Journal of Physical Chemistry B, 125 (2021) 7717-7731.
[65] J. Wainwright, G. Hobbs, I. Nakouti, Archives of Microbiology, 203 (2021) 5899-5906.
[66] S. Manuse, Y. Shan, S. J. Canas-Duarte, S. Bakshi, W. S. Sun, H. Mori, J. Paulsson, K. Lewis, PLOS biology. 19 (2021) e3001194.
[67] J. Yeom, E. A. Groisman, Science Signaling, 14 (2021) eabc4235.
[68] C. Rezzoagli, E. T. Granato, R. Kummerli, Journal of medical microbiology, 69 (2020) 147.
[69] N. Bhargava, P. Sharma, N. Capalash, Infection and immunity, 82 (2014) 3417-3425.
[70] M. Sultan, R. Arya, K. K. Kim, International Journal of Molecular Sciences, 22 (2021) 12152.
[71] G. M. Denning, L. A. Wollenweber, M. A. Railsback, C. D. Cox, L. L. Stoll, B. E. Britigan, Infection and immunity, 66 (1998) 5777-5784.
[72] G. W. Lau, H. Ran, F. Kong, D. J. Hassett, D. Mavrodi, Infection and immunity, 72 (2004) 4275-4278.
[73] H. Ogawara, The Journal of Antibiotics, 74 (2021) 24-41.
[74] R. C. Allen, R. Popat, S. P. Diggle, S. P. Brown, Nature Reviews Microbiology, 12 (2014) 300-308.
[75] A. Panáček, L. Kvítek, M. Smékalová, R. Večeřová, M. Kolář, M. Röderová, F. Dyčka, M. Šebela, R. Prucek, O. Tomanec, Nature nanotechnology, 13 (2018) 65-71.
[76] N. El Kary, E. El Rassy, N. Azar, J. Choucair, American Journal of Infection Control, 44 (2016) 1736-1737.
[77] K. M. Jørgensen, T. Wassermann, P. Ø. Jensen, W. Hengzuang, S. Molin, N. Høiby, O. Ciofu, Antimicrobial agents and chemotherapy, 57 (2013) 4215-4221.
[78] T. C. Barrett, W. W. Mok, A. M. Murawski, M. P. Brynildsen, Nature communications, 10 (2019) 1-11.
[79] L. Goldberg, S. Filippos' iants, I. Belova, T. Vertogradova, N. Shepelevtseva, Antibiotiki, 24 (1979) 60-67.
[80] K. M. Tsoi, S. A. MacParland, X. Z. Ma, V. N. Spetzler, J. Echeverri, B. Ouyang, S. M. Fadel, E. A. Sykes, N. Goldaracena, J. M. Kaths, Nature materials, 15 (2016) 1212-1221.
[81] C. N. Loynachan, A. P. Soleimany, J. S. Dudani, Y. Lin, A. Najer, A. Bekdemir, Q. Chen, S. N. Bhatia, M. M. Stevens, Nature nanotechnology, 14 (2019) 883-890.
[82] D. W. Essar, L. Eberly, A. Hadero, I. Crawford, Journal of bacteriology, 172 (1990) 884-900.

1. A nanocluster comprising a metallic core conjugated to a nucleotide, wherein the metallic core has a diameter of less than 10 nm.

2. The nanocluster of claim 1, wherein the nucleotide is adenosine triphosphate (ATP) or a phosphorothioate analog, a deoxyribonucleotide analog, a 7-deaza purine nucleotide analog, or a phosphomethylphosphonic acid adenylate ester thereof.

3. The nanocluster of claim 2, wherein the phosphorothioate analog is ATPαS, ATPβS, or ATPγS, wherein the deoxyribonucleotide analog is deoxyadenosine triphosphate (dATP), wherein the 7-deaza purine nucleotide analog is 7-deazaadenosine-5′-triphosphate (7-deaza-ATP), or wherein the phosphomethylphosphonic acid adenylate ester is β,γ-methyleneadenosine 5′-triphosphate (AMP-PCP).

4-6. (canceled)

7. The nanocluster of claim 1, wherein the diameter of the nanocluster ranges from about 1 nm to about 2 nm as measured using transmission electron microscopy.

8. The nanocluster of claim 1, wherein the metallic core comprises a noble metal.

9. The nanocluster of claim 8, wherein the metallic core is a gold metallic core.

10. (canceled)

11. The nanocluster of claim 1, wherein the nanocluster is linked to an internalization sequence, a protein transduction domain, or a cell penetrating peptide.

12. A composition comprising the nanocluster of claim 1 and a pharmaceutically acceptable excipient, a pharmaceutically acceptable carrier, an antibiotic, or any combination thereof.

13-16. (canceled)

17. A method of treating an infection in a subject, the method comprising administering a therapeutically effective amount of the composition of claim 12 to the subject.

18. The method of claim 17, wherein the infection is a bacterial infection.

19. The method of claim 18, wherein the bacterial infection is a Staphylococcus aureus, Klebsiella pneumoniae, Pseudomonas aeruginosa, or Escherichia coli infection.

20. The method of claim 17, further comprising administering a therapeutically effective amount of at least one antibiotic in combination with the composition.

21. The method of claim 17, wherein the infection is a chronic infection.

22. The method of claim 17, wherein the infection is an ear infection, a cutaneous infection, a lung infection, a catheter-associated urinary tract infection, or a gastrointestinal infection.

23. The method of claim 17, wherein the infection is associated with formation of a bacterial biofilm in the subject.

24-25. (canceled)

26. The method of claim 17, wherein the infection comprises pathogenic bacteria that are resistant to one or more antibiotics.

27-33. (canceled)

34. The method of claim 17, wherein multiple cycles of treatment are administered to the subject.

35. The method of claim 17, wherein the composition is administered intravenously, subcutaneously, by inhalation, topically, or locally at the site of infected tissue.

36. (canceled)

37. The method of claim 35, wherein the infection is an ear infection, and the composition is administered locally into the ear canal.

38-39. (canceled)

40. A method of eradicating bacteria in a biofilm or dormant bacteria comprising persister cells, the method comprising contacting the biofilm or dormant bacteria comprising persister cells with an effective amount of the nanocluster of claim 1.

41. The method of claim 40, further comprising contacting the biofilm or dormant bacteria comprising persister cells with an effective amount of at least one antibiotic.

42. The method of claim 40, wherein the biofilm or dormant bacteria are is on a medical device, a personal hygiene article, toiletry, cosmetic, disinfectant, cleaning solution, or in a water treatment or distribution system.

43-44. (canceled)

45. The method of claim 40, wherein the dormant bacteria are in a biofilm, in a liquid culture, or on an inanimate surface.

46. A method of inhibiting a virulence factor of a baterium, the method comprising contacting the bacterium with an effective amount of the nanocluster of claim 1.

47-48. (canceled)
